(19) United States
(12) Patent Application Publication
(10) Pub. No.: US 2024/0336895 A1
(43) Pub. Date: Oct. 10, 2024

(54) METHODS FOR RAPIDLY INFILTRATING 3D SCAFFOLDS WITH CELLS

(71) Applicant: Spiderwort Inc., Ottawa (CA)

(72) Inventors: Andrew E. PELLING, Ottawa (CA); Matthew LOURENCO, Mississauga (CA); Ryan Hickey, Ottawa (CA); Paula Cristina de Sousa Faria Tischer, Curitiba (BR); Anna CANTO, Carleton Place (CA); Joshua SALAMUN, Ottawa (CA); Colin RUSSELL, Ottawa (CA)

(21) Appl. No.: 18/293,128

(22) PCT Filed: Jul. 29, 2022
(86) PCT No.: PCT/CA2022/051169

§ 371 Date: Jan. 29, 2024

Related U.S. Application Data
(60) Provisional application No. 63/227,729, filed on Jul. 30, 2021.

(51) Int. Cl. C12N 5/077; A23J 3/22; A23L 5/49; A23L 13/00; A23L 17/00; A23L 19/00; C12N 5/04

(52) U.S. Cl. CPC C12N 5/0658; A23J 3/227; A23L 5/49; A23L 13/00; A23L 17/00; A23L 19/09; C12N 5/04; C12N 2513/00; C12N 2533/70; C12N 2533/90; C12N 2537/10

(57) ABSTRACT

Disclosed herein is a method of preparing a seeded biomaterial scaffold. The method comprises combining a biomaterial scaffold and a plurality of cells to provide a mixture; and applying a pressure to the mixture to thereby cause the plurality of cells to infiltrate the scaffold, thereby forming the seeded biomaterial scaffold.

### TECHNICAL FIELD

The present disclosure generally relates to 3D cell culturing techniques. More specifically, the present disclosure relates to methods for rapidly infiltrating 3D scaffolds with cells.

### BACKGROUND

3D cell culturing generally involves culturing cells using a 3D scaffold. 3D cell cultures have a variety of uses across a number of different industries such as the pharmaceutical, medical device, cosmetic, and food industries.

With respect to the food industry, 3D cell cultures may be used to produce what are commonly referred to as “lab-grown” or “cultured” meat products. Conventionally, 3D cell cultures for meat products are produced using the same standard processes as those used to produce cultured biomaterial scaffolds for tissue engineering applications. However, such processes are not well adapted for the food industry and present a number of disadvantages. For example, the cell culture media used to proliferate stem cells into muscle or fat tissues contain fetal bovine serum (FBS). As well, for continued cell proliferation, the cell culture media has to be renewed daily. Thus, such processes consume a considerable amount of FBS, which bears a significant cost to produce and represent an ethical concern.

Further, in addition to renewing the FBS-containing cell culture media daily, for continued cell proliferation, the scaffolds must be moved into new plates every two weeks for up to twelve weeks. As a result, the conventional processes for producing meat products using 3D scaffolds are very labour-intensive, which may also increase the cost of the resulting cultured meat product.

Furthermore, as cells proliferate within scaffolds, the cells located within the central portions of the scaffold begin to suffer from oxygen and/or nutrient deprivation due to the diffusion limits of the scaffold and cells contained thereon. The oxygen and/or nutrient deprivation leads to cell necrosis in the central portions of the scaffold, resulting in a “necrotic core”. As described above, the proliferation of cells is a long process (e.g. up to twelve weeks) and, as a result, it may be difficult to prevent the development of the necrotic core. The cell necrosis issue is especially pertinent for larger and thicker scaffolds—i.e. those required for cultured meat production.

### SUMMARY

The present disclosure generally relates to methods for producing seeded 3D biomaterial scaffolds. In particular, the present disclosure relates to improved methods for producing seeded 3D biomaterial scaffolds that may be used to produce cultured meat products therefrom.

One aspect of the present disclosure relates to a method of preparing a seeded biomaterial scaffold, the method comprising: combining a biomaterial scaffold and a plurality of cells to provide a mixture; and applying a pressure to the mixture to thereby cause the plurality of cells to homogeneously distributed throughout the scaffold, thereby forming the seeded biomaterial scaffold.

Another aspect of the present disclosure relates to a seeded biomaterial scaffold produced by the methods described herein.

Yet another aspect of the present disclosure relates to the use of the seeded biomaterial scaffold described herein for the production of a cultured meat product.

Other aspects and features of the methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a photograph of the biomaterial scaffolds of Example 1, wherein FIG. 1A shows the Scaffold 4 of Example 1, FIG. 1B shows the Scaffold 3 of Example 1, FIG. 1C shows the Scaffold 2 of Example 1, and FIG. 1D shows the Scaffold 1 of Example 1;

FIG. 2 shows microscopy images of one of the samples of Scaffold 1 of Example 1, wherein FIG. 2A shows a microscopy image of a top side of the Scaffold 1, FIG. 2B shows a microscopy image of an interior of the Scaffold 1, and FIG. 2C shows a microscopy image of a bottom side of the Scaffold 1;

FIG. 3 shows microscopy images of one of the samples of Scaffold 2 of Example 1, wherein FIG. 3A shows a microscopy image of a top side of the Scaffold 2, FIG. 3B shows a microscopy image of an interior of the Scaffold 2, and FIG. 3C shows a microscopy image of a bottom side of the Scaffold 2;

FIG. 4 shows microscopy images of one of the samples of Scaffold 3 of Example 1, wherein FIG. 4A shows a microscopy image of a top side of the Scaffold 3, FIG. 4B shows a microscopy image of an interior of the Scaffold 3, and FIG. 4C shows a microscopy image of a bottom side of the Scaffold 3;

FIG. 5 shows microscopy images of one of the samples of Scaffold 4 of Example 1, wherein FIG. 5A shows a microscopy image of a top side of the Scaffold 4, FIG. 5B shows a microscopy image of an interior of the Scaffold 4, and FIG. 5C shows a microscopy image of a bottom side of the Scaffold 4;

FIG. 6 shows a microscopy image of a longitudinal section of one of the samples of Scaffold 1 of Example 1;

FIG. 7 shows a microscopy image of a longitudinal section of one of the samples of Scaffold 3 of Example 1;

FIG. 8 shows microscopy images of Scaffolds 5 and 6 and the Negative Control of Example 2, wherein FIG. 8A shows a microscopy image of Scaffold 5, FIG. 8B shows a microscopy image of Scaffold 6, and FIG. 8C shows a microscopy image of the Negative Control;

FIG. 9 shows microscopy images of the meat standards of Example 2, wherein FIG. 9A shows a microscopy image of one of the tuna samples, FIG. 9B shows a microscopy image of one of the scallop samples, and FIG. 9C shows a microscopy image of one of the beef samples;

FIG. 10 shows microscopy images of the biomaterial scaffold of Example 3, wherein FIG. 10A shows a microscopy image of an exterior of the biomaterial scaffold, and FIG. 10B shows a microscopy image of an interior of the biomaterial scaffold;

FIG. 11 shows microscopy images of the further stained biomaterial scaffold of Example 3, wherein FIG. 11A shows a microscopy image of an exterior of the biomaterial scaffold, and FIG. 11B shows a microscopy image of an interior of the biomaterial scaffold;

FIG. 12 shows a microscopy image of the biomaterial scaffold of Example 4, wherein FIG. 12A is a microscopy image of a textured edge of the scaffold, FIG. 12B is a microscopy image of an interior of the scaffold, and FIG. 12C is a microscopy image of a smooth edge of the scaffold;

FIG. 13 shows microscopy images of the biomaterial scaffolds as described in Example 8, wherein FIG. 13A is a microscopy image of a sample produced by the process of Example 7, FIG. 13B is a microscopy image of a sample produced by the process of Example 6, and FIG. 13C is a microscopy image of a sample produced by the standard process;

FIG. 14 shows the FTIR results of the samples imaged in FIG. 13;

FIG. 15 shows the FTIR results of the samples imaged in FIG. 13 in addition to decellularized apple and raw apple;

FIG. 16 shows single particles of the biomaterial scaffolds as described in Example 8, wherein FIG. 16A shows single particles of a sample produced by the process of Example 7, FIG. 16B shows a single particle of a sample produced by the process of Example 6, and

FIG. 16C shows single particles of a sample produced using the standard process;

FIG. 17 shows the particle size distributions for the biomaterial scaffolds as described in Example 8, wherein FIG. 17A is a histogram illustrating the number of particles in specific size ranges for the sample produced by the process of Example 7, FIG. 17B is a histogram illustrating the number of particles in specific size ranges for the sample produced by the process of Example 6, and FIG. 17C is a histogram illustrating the number of particles in specific size ranges for the sample produced by the standard process;

FIG. 18 shows microscopy images for the mercerized samples described in Example 9, wherein FIG. 18A shows a microscopy image of the apples mercerized using 15% hydrogen peroxide stock solution, and FIG. 18B shows a microscopy image of the apples mercerized using 30% hydrogen peroxide stock solution;

FIG. 19 shows particle size distributions for each of the mercerized samples described in Example 9, wherein FIG. 19A shows the particle distribution of the sample prepared using the 10% sodium bicarbonate and 15% hydrogen peroxide stock solution, FIG. 19B shows the particle distribution of the sample prepared using the 10% sodium bicarbonate and 30% hydrogen peroxide stock solution, and FIG. 19C shows the particle distribution of the sample prepared using the 1M NaOH and 30% hydrogen peroxide stock solution;

FIG. 20 shows the FTIR results for each of the mercerized samples described in Example 9;

FIG. 21 shows photographs of cooked biomaterial scaffolds described in Example 12, wherein FIG. 21A shows the sous vide samples, FIG. 21B shows the pan-fried samples, and FIG. 21C shows the baked samples;

FIG. 22 shows the results of the taste-testing of the cooked biomaterial scaffolds described in Example 14, wherein FIG. 22A shows the distribution of descriptors used to describe the flavour of the samples, and FIG. 22B shows the distribution of descriptors used to describe the texture of the samples;

FIG. 23 shows photographs of the samples prepared for the “Colour Stability during Freeze-drying” study of Example 15, wherein FIG. 23A shows the samples prior to freeze-drying and FIG. 23B shows the samples after freeze-drying;

FIG. 24 shows a photograph of the samples prepared for the “Colour Stability in Water and Heat” study of Example 15;

FIG. 25 shows photographs of the samples prepared for the “Colour Stability after Light Exposure” study of Example 15, wherein FIG. 25A shows the two halves of the sample before treatment, FIG. 25B shows the two halves after day 2, FIG. 25C shows the two halves after day 3, FIG. 25D shows the two halves after day 4;

FIG. 26 shows photographs of the samples prepared for the “Colour Stability using different concentrations of Mercerized Apple and different durations of Crosslinking” study of Example 15, wherein FIG. 26A shows the biopsy punches of sample (1), FIG. 26B shows the biopsy punches of sample (2), and FIG. 26C shows the biopsy punches of sample (3);

FIG. 27 shows photographs of imitation foods prepared in Example 16, wherein FIG. 27A shows an imitation fish stick, FIG. 27B shows an imitation scallop, and FIG. 27C shows a layer of the imitation fish stick peeled back;

FIG. 28 shows microscopy images of Sample 1 of Example 17, wherein FIG. 28A shows a microscopy image of the top side of the sample at 0.7×, FIG. 28B shows a microscopy image of the top side of the sample at 1.6×, FIG. 28C shows a microscopy image of the bottom side of the sample at 0.7×, and FIG. 28D shows a microscopy image of the bottom side of the sample at 1.6×;

FIG. 29 shows microscopy images of Sample 2 of Example 17, wherein FIG. 29A shows a microscopy image of an edge of the sample at 0.7×, FIG. 29B shows a microscopy image of the centre of the sample at 0.7×, FIG. 29C shows a microscopy image of the edge of the sample at 1.0×, and FIG. 29D shows a microscopy image of the centre of the sample at 1.0×;

FIG. 30 shows microscopy images of Sample 3 of Example 17, wherein FIG. 30A shows a microscopy image of a top surface of the sample 3A (not dyed with beetroot powder) at 1.0×, FIG. 30B shows a microscopy image of a side of the sample 3B after lyophilization at 1.6×, and FIG. 30C shows a microscopy image of a corner of the sample 3B at 1.6×;

FIG. 31 shows photographs of the portions of the first sample of Example 18, wherein FIG. 31A shows a photograph of a side of the portion crosslinked for 1 hour, FIG. 31B shows a photograph of a side of the portion crosslinked for 1 hour after cooking, FIG. 31C shows a photograph of a side of the portion crosslinked for 24 hours, and FIG. 31D shows a photograph of a side view of the portion crosslinked for 24 hour after cooking;

FIG. 32 shows photographs of the portions of the second sample of Example 18, wherein FIG. 32A shows a photograph of a top side of the second sample after crosslinking, and FIG. 32B shows a photograph of the top side of the second sample after pan-frying;

FIG. 33 shows a photograph of an imitation pulled pork sandwich as described in Example 20;

FIG. 34 shows imitation foie gras as described in Example 20, wherein FIG. 34A shows a photograph of the plated formulation F1, FIG. 34B shows a photograph of the plated formulation F2, FIG. 34C shows a photograph of the formulation F3 on a cracker, and FIG. 34D shows a photograph of the formulations F4 (left) and F5 (middle) compared to actual foie gras (right);

FIG. 35 shows imitation fish sticks as described in Example 20, wherein FIG. 35A shows a photograph of the first formulation cut open and FIG. 35B shows a photograph of the second formulation cut open;

FIG. 36 shows microscopy images of a seeded biomaterial scaffold stained with hematoxylin and eosin (HE) as described in Example 21, wherein FIG. 36A shows the HE-stained seeded biomaterial scaffold at a magnification level of 2.5×, and FIG. 36B shows the HE-stained seeded biomaterial scaffold at a magnification level of 10×;

FIG. 37 shows microscopy images of a seeded biomaterial scaffold stained with Masson's Trichrome (MT) as described in Example 21, wherein FIG. 37A shows the MT-stained seeded biomaterial scaffold at a magnification level of 2.5×, and FIG. 37B shows the MT-stained seeded biomaterial scaffold at a magnification level of 10×;

FIG. 38 shows microscopy images of a beef sample cut perpendicularly relative to the fibers thereof stained with HE as described in Example 21, wherein FIG. 38A shows the HE-stained beef sample at a magnification level of 2.5×, and FIG. 38B shows the HE-stained beef sample at a magnification level of 10×;

FIG. 39 shows microscopy images of a beef sample cut perpendicularly relative to the fibers thereof stained with MT as described in Example 21, wherein FIG. 39A shows the MT-stained beef sample at a magnification level of 2.5×, and FIG. 39B shows the MT-stained beef sample at a magnification level of 10×;

FIG. 40 shows microscopy images of a scallop sample cut longitudinally relative to the fibers thereof stained with HE as described in Example 21, wherein FIG. 40A shows the HE-stained scallop sample at a magnification level of 2.5×, and FIG. 40B shows the HE-stained scallop sample at a magnification level of 10×;

FIG. 41 shows microscopy images of a scallop sample cut longitudinally relative to the fibers thereof stained with MT as described in Example 21, wherein FIG. 41A shows the MT-stained scallop sample at a magnification level of 2.5×, and FIG. 41B shows the MT-stained scallop sample at a magnification level of 10×;

FIG. 42 shows microscopy images of a scallop sample cut perpendicularly relative to the fibers thereof stained with HE as described in Example 21, wherein FIG. 43A shows the HE-stained scallop sample at a magnification level of 2.5×, and FIG. 43B shows the HE-stained scallop sample at a magnification level of 10×;

FIG. 43 shows microscopy images of a scallop sample cut perpendicularly relative to the fibers thereof stained with MT as described in Example 21, wherein FIG. 43A shows the MT-stained scallop sample at a magnification level of 2.5×, and FIG. 43B shows the MT-stained scallop sample at a magnification level of 10×;

FIG. 44 shows microscopy images of a tuna sample cut perpendicularly relative to the fibers thereof stained with HE as described in Example 21, wherein FIG. 44A shows the HE-stained tuna sample at a magnification level of 2.5×, and FIG. 44B shows the HE-stained tuna sample at a magnification level of 10×;

FIG. 45 shows microscopy images of a tuna sample cut perpendicularly relative to the fibers thereof stained with MT as described in Example 21, wherein FIG. 45A shows the MT-stained tuna sample at a magnification level of 2.5×, and FIG. 45B shows the MT-stained tuna sample at a magnification level of 10×;

FIG. 46 shows photographs of a vegan sashimi sample incorporating therein a seeded biomaterial scaffold produced using a method according to an embodiment of the present disclosure, wherein FIG. 46A shows a photograph of a cross-section of the vegan sashimi before cooking via sous vide, and FIG. 46B shows a photograph of the cooked vegan sashimi.

FIG. 47 shows the preparation (cell addition, assembling, and cooking simulating the Sous Vide) of the cell-based salmon prototype formulation.

FIG. 48 shows the preparation (cell addition, assembling, cooking simulating the Sous Vide, and tasting) of the cell-based salmon prototype formulation.

FIG. 49 shows the preparation of the cell-based salmon prototype, which is ready for tasting.

FIG. 50 shows the tasting of the cell-based salmon prototype.

FIG. 51A shows the C2C12 cells line proliferating in DMEM, and FIG. 51B shows the suspension of cells after 18 days of proliferation.

FIG. 52 shows WBF-material infiltrated with Chinook salmon cells after the vacuum process.

FIG. 53 shows C2C12 cells line inoculated in the WBF-material in formalin.

FIG. 54 shows the histology of the cell-based salmon prototype infiltrated with C2C12 cells (surface of the sample) at 10× magnification, HE.

FIG. 55 shows the histology of the cell-based salmon prototype infiltrated with C2C12 cells (surface of the sample) at 10× magnification, HE.

FIG. 56 shows the histology of the Control (without cells), the surface and middle of the plant-based salmon prototype (Left and Right respectively) at 10× magnification, HE.

FIG. 57 shows the starting material for the microscopy analysis with the cells suspended in PFA (seeded with Chinook salmon cells).

FIG. 58 shows the results of the microscopy analysis of seeded scaffold (seeded with Chinook salmon cells) stained with Hoescht (1:1000) under DAPI filters and 10× magnification and scale: 100 μm, where Cyan represents nuclei and magenta represents scaffold.

### DETAILED DESCRIPTION

The present disclosure generally relates to methods for producing seeded 3D biomaterial scaffolds. In particular, the present disclosure relates to improved methods for producing seeded biomaterial scaffolds that may be used to produce cultured meat products therefrom. The disclosure also relates to rapid infiltration of large scale biomaterial scaffolds, and in certain embodiments can facilitate doing so with a high density of cells.

In more detail, the methods of the present disclosure may provide a number of advantages over the conventional methods for producing seeded 3D scaffolds to be used for meat products. For example, the methods of the present disclosure are capable of rapidly infiltrating cells within scaffolds. Conventional methods involve proliferating cells within the scaffold over the course of 2 to 12 weeks, and longer in some circumstances, in order to achieve highly infiltrated scaffolds. In contrast, the methods of the present disclosure may rapidly infiltrate cells within a scaffold in about 1 week or less. As will be appreciated, faster cell infiltration within the biomaterial scaffold may reduce the likelihood of cell necrosis and may, in turn, reduce the likelihood of the seeded biomaterial scaffold developing a necrotic core.

Further, cells may be infiltrated within the scaffold in a minimum amount of steps. As previously described herein, for continued proliferation, conventional methods generally involve renewing the cell culture media daily as well as moving the scaffolds to new plates about every two weeks, to obtain a high degree of infiltration and acceptable cell density. Thus, the methods of the present disclosure may be less labour-intensive and simpler to perform.

Furthermore, the methods of the present disclosure may also use significantly less fetal bovine serum (FBS) than conventional methods. Conventional methods generally require that the cell culture media, which contains FBS, be replaced daily. As discussed above, FBS is considerably expensive and thus significantly increases the cost of those methods. The methods of the present disclosure do not require the daily replacement of cell culture media and therefore require less FBS than the conventional methods. As a result, the methods may be substantially less expensive to perform than the conventional methods.

Further still, the methods of the present disclosure may be performed “kitchen-safe”. That is, the reagents, the experimental conditions, and the equipment used may be selected such that they are suitable for use in a commercial kitchen and are compliant with industry regulations.

Additional advantages will be discussed below and will be readily apparent to those of ordinary skill in the art upon reading the present disclosure.

According to an embodiment, the present disclosure relates to method of preparing a seeded biomaterial scaffold, the method comprising: combining a biomaterial scaffold and a plurality of cells to provide a mixture; and applying a pressure to the mixture to thereby cause the plurality of cells to infiltrate the scaffold, thereby forming the seeded biomaterial scaffold.

As will be appreciated, in some embodiments, a biomaterial scaffold may be a 3D support, an underlying architecture, and/or an infrastructure for cells to infiltrate and/or proliferate. The biomaterial scaffold may have a rigid shape, or may be in the form of a gel (e.g. a hydrogel or an aerogel) or a paste.

The biomaterial scaffold used in the methods of the present disclosure may be produced using any suitable technique. For example, in some embodiments, the biomaterial scaffolds may be produced using the process outlined in U.S. Patent Application No. 63/107,226, the disclosure of which is hereby incorporated by reference in its entirety. In some embodiments, the biomaterial scaffold may be produced by using pressure driven cell infiltration along with crosslinking the mixture of the biomaterial scaffold and the cells in a sealed/molded environment.

Further, in the context of the present disclosure, the scaffolds are biomaterial scaffolds in that they are derived from a biomaterial such as a suitable plant or fungal tissue. As will be understood, unless otherwise indicated, the meaning/definition of plant and fungi kingdoms used herein is based on the Cavalier-Smith classification (1998).

In some embodiments, the plant or fungal tissue from which the biomaterial scaffold may be derived comprises an apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesil) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, a rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, a pear (Pomaceous) tissue, a heart of the palm (Bactris gasipaes) tissue, an artichoke (Cynara cardunculus var. scolymus) tissue, a lotus roots (Nelumbo nucifera), banana blossom (Musa acuminata), Bamboo shoot (Bambusa vulgaris and Phyllostachys edulis), or any combination thereof.

In some embodiments, the plant or fungal tissue may be genetically altered via direct genome modification or through selective breeding, to create an additional plant or fungal architecture which may be configured to physically mimic a tissue and/or to functionally promote a target tissue effect.

In some embodiments, the plant or fungal tissue may be decellularized plant or fungal tissue. The decellularized plant or fungal tissue lacks cellular materials and nucleic acids of plant or fungal cells while maintaining the 3D structure thereof substantially intact. As will be understood, cellular materials and nucleic acids may include intracellular contents such as cellular organelles (e.g. chloroplasts, mitochondria), cellular nuclei, cellular nucleic acids, and cellular proteins. These may be substantially removed, partially removed, or fully removed from the scaffold biomaterial. It will recognized that trace amounts of such components may still be present in the decellularized plant or fungal tissues described herein.

In some embodiments, the plant or fungal tissue may be decellularized by mixing the plant or fungal tissue with a detergent or a surfactant. Non-limiting examples of suitable detergents may include sodium dodecyl sulphate (SDS)/Sodium Lauryl Sulphate (SLS) Triton X, ethylenediamine (EDA), alkaline detergents, acidic detergents, ionic detergents, non-ionic detergents, and zwitterionic detergents. In an embodiment, the plant or fungal tissue may be decellularized by mixing with a SDS/SLS solution having an SDS/SLS concentration of between 0.01 to 10%, for example about 0.1% to about 1%, or, for example, about 0.1% SDS/SLS or about 1% SDS/SLS, in a solvent such as water, ethanol, or another suitable organic solvent. In such embodiments, it may be desired to remove residual SDS/SLS (if present) prior to use of the decellularized plant or fungal tissue. Residual SDS/SLS may be removed using, for example, a divalent salt solution. The divalent salt solution may be an MgCl₂ solution, a CaCl₂ solution, or the like. The divalent salt solution may comprise the divalent salt at a concentration of about 50 mM to about 150 mM (e.g. about 100 mM). In some embodiments, after removal of the residual SDS/SLS solution, the decellularized plant or fungal tissue may be washed or incubated in dH₂O to remove any remaining solubilized SDS/SLS and/or SDS/SLS micelles past the cloud point.

In some embodiments, the plant or fungal tissue may be a mercerized plant or fungal tissue. As will be appreciated, mercerization disassembles the plant or fungal tissue into tissue/cellular components. Conventional mercerization techniques typically involve the use of a solution capable of osmotic shock and/or disruption of hydrogen bonding and/or polymer crystal structure so as to extract intact tissue structures from the plant or fungal tissue. In some embodiments, the base solution may be a NaOH solution, a KOH solution, a Ca(OH)2 solution or the like having a concentration of about 1M, or NaHCO₃ solution in a concentration of about 10%.

After treatment with the base, conventional mercerization techniques typically involve bleaching the material (e.g. the plant or fungal tissue). The bleaching of the material may be performed using a peroxide such as hydrogen peroxide. Conventionally, the hydrogen peroxide is used at a stock solution concentration of from about 6% to about 30% stock solution.

Further, in some embodiments, the mercerization may be performed with heating. In such embodiments, the mixture of the regent(s) and the plant or fungal tissue being mercerized may be heated to from about 40° C. to 80° C.

As described above, in some embodiments, the methods of the present disclosure may be performed entirely in a non-lab setting such as a kitchen and therefore may be “kitchen-safe”. While the above discussed conventional decellularization and mercerization techniques use reagents that may be generally recognized as safe (GRAS), the reagents and the concentrations thereof may not be particularly suitable for use in, for example, a kitchen setting. Thus, in some embodiments, the mercerization process may be performed using a base such as a carbonate solution. In such embodiments the base may be, for example, a bicarbonate solution such as a sodium bicarbonate solution. The bicarbonate solution may include the bicarbonate in a concentration of greater than or about 10% (w/v). Further, if bleaching is required, a hydrogen peroxide solution may still be used however at a concentration of less than 30% (w/w), such as about 15% or about 9% (w/w) for the stock solutions. Such embodiments may be particularly suited for kitchen-based applications, as the mercerization may be performed without, for example, the personal protective equipment (PPE) required to handle reagents such as 1M NaOH or 30% hydrogen peroxide stock solution especially if heating is performed during the mercerization process.

As will be described in detail below, the methods of the present disclosure may comprise crosslinking the mixture of the biomaterial scaffold and the cells. Thus, in some embodiments, the biomaterial scaffold may be crosslinkable. The biomaterial scaffold may be crosslinkable due to the materials from which it is derived. For example, if the biomaterial scaffold is derived from a plant tissue, the biomaterial scaffold may comprise cellulose or a cellulose derivative such as methylcellulose, carboxymethylcellulose, hydroxypropylcellulose, hydroxypropyl methyl cellulose, hydroxyethyl methyl cellulose, ethyl cellulose and dissolved or regenerated cellulose, which are crosslinkable. However, in some embodiments, it may be desirable to add additional crosslinkable components to the biomaterial scaffold. For example, in some embodiments, the biomaterial scaffold may incorporate different classes of food hydrocolloids comprising one or more crosslinkable components such as plant-derived hydrocolloids (including but not limited to plant exudates; such as acacia gum, gum arabic, tragacanth, khaya gum, karaya gum, ghatti gum, pectin, inulin, chicle gum, konjac glucomannan; seed gums such as guar gum, locust bean gum, fenugreek gum, cassia seed gum, basil seed gum, mesquite seed gum, oat gum, lesquerella, fendleri gum, rye gum, Psyllium, premcem gum, starch, amylase, cellulose, tamarind seed gum, seaweed—agar-agar, carrageenan, alginic acid, sodium alginate, furcellaran, ulvan, flucoidan, laminarin, red alga, xylan) animal-derived hydrocolloids (including but not limited to gelatin, chitin, and chitosan), hydrocolloids from microbial sources—fermentation (microbial exudates—xanthan, dextran, curdlan, scleroglucan, gellan gum, pullulan, tara guma, spruce gum, and baker's yeast glycan), and chemically modified plant-derived hydrocolloids-synthetic gums (including but not limited to modified starch—hetastarch, starch acetates, startch phosphates) hyaluronic acid, elastin, fibrin, fibrinogen, or the like, or any combination thereof. The aforementioned components (including proteins) can be cross-linked using chemical, physical, or enzymatic techniques, for example, using glutaraldehyde, glyoxal, genipin, diimidoesters-dimethyl suberimidate, 3,3′-dithiobispropionimidate, sorbitol, glycerol, hexamethylene diisocyanate (HMDC), calcium chloride, monovalent ions such as H+, Na+, K+, Cs+, Rb+, and I−, multivalent ions such as Mg2+, Ca2+, Ba2+, Fe2+, Cu2+, Zn2+, Fe3+ and A13+, divalent ion salts, acids (such as citric acid, tannic acid, malic acid, and glutamic acid), enzymes (such as tranglutaminase, oxidoreductases), phenolic acids, flavonoids, glucono delta lactone, high pressure, irradiation, optical radiation, ionizing radiation, and the like. The one or more crosslinkable components may be included in any suitable amount.

As described above, in the methods of the present disclosure, the biomaterial scaffold is combined with a plurality of cells to provide a mixture. The cells may be combined with the biomaterial scaffold using any suitable technique. For example, in some embodiments, the cells and the biomaterial scaffold may be mixed and placed in the same container (e.g. a vacuum-sealable container).

The types of cells used may be selected depending on the application of the resulting seeded biomaterial scaffold. In certain embodiments, the biomaterial scaffold may be seeded with one or more cells selected from fibroblasts, myofibroblasts, neurons, dorsal root ganglion cells, neuronal structures such as axons, neural precursor cells, neural stem cells, glial cells, endogenous stem cells, neutrophils, mesenchymal stem cells, satellite cells, myoblasts, myotubes, muscle progenitor cells, adipocytes, preadipocytes, chondrocytes, osteoblasts, osteoclasts, pre-osteoblasts, tendon progenitor cells, tenocytes, periodontal ligament stem cells, endothelial cells, or any combinations thereof. In further embodiments, such as where food product applications are of interest, the one or more cells may comprise muscle cells, fat cells, connective tissue cells (i.e. fibroblasts), cartilage, bone, epithelial, or endothelial cells, or any combinations thereof. For example, if the seeded biomaterial scaffold is to be used in the food industry as a lab-grown or cultured meat product, the cells may be stem cells such as pluripotent stem cells, induced pluripotent stem cells, multipotent stem cells, and unipotent stem cells. In yet further embodiments, the cells may be those of the animal of the imitation meat product that the seeded biomaterial scaffold is intended to recreate.

In some embodiments, the cells may be provided in a cell culture media. The cell culture media may be selected based on the types of cells used and/or the intended use of the resulting seeded biomaterial scaffold. For example, in some embodiments, the cell culture media may comprise a medium such as Dulbecco's Modified Eagle Medium (DMEM).

The cell culture media may also comprise one or more additives to support or encourage the growth or proliferation of the cells within the biomaterial scaffold. In some embodiments, the cell culture media may comprise growth factors such as those found in fetal bovine serum (FBS) or another animal-derived serum, or a combination of single specific growth factors mixed together. In such embodiments, the growth factor may be included at a concentration of greater than 0.1%. In some embodiments, the growth factor may be included at a concentration of about 1% to about 20%. In a particular embodiment, the growth factor may be included at a concentration of about 10%.

The cell culture media may comprise one or more antibiotics. The one or more antibiotics may be included to prevent the growth of, for example, potentially harmful microbes during seeding and/or proliferation of the cells within the biomaterial scaffold. The inclusion of one or more antibiotics may be particularly useful if the seeded biomaterial scaffold is to be used to produce an edible cultured meat product. The one or more antibiotics may be any suitable antibiotics. For example, in some embodiments, one or more antibiotics may comprise penicillin-streptomycin. Further, the one or more antibiotics may be included in any amount at which they are capable of preventing the growth of the potentially harmful microbes. In some embodiments, each of the one or more antibiotics may be included at a concentration of about 0.01% to 5% or more or less as needed. In a particular embodiment, each of the one or more antibiotics may be included at a concentration of about 1%.

As described above, once the biomaterial scaffold and plurality of cells are combined, a pressure is applied thereto in order to cause the polarity of cells to be homogeneously distributed throughout the scaffold. The pressure may be a positive pressure or a decrease in pressure. Without being bound to any particular theory, it is thought that the application of pressure causes the cells to spread within, or infiltrate, the biomaterial scaffold. In certain non-limiting embodiments, a decrease in pressure may be applied (i.e. decreased below atmospheric pressure conditions) while maintaining cell viability. For example, this may involve ranges from about 0 to about 101.3 kPa below atmospheric pressure, more particularly from about 0 to about 100 kPa below atmospheric pressure. In other applications a positive pressure may be utilized (i.e. above atmospheric pressure conditions) while maintaining cell viability. For example, this may involve ranges from about 0.001 to about 900 MPa, more particularly from about 100 to about 700 MPa above atmospheric pressure.

In embodiments where the pressure is a decrease in pressure relative to atmospheric pressure, the application of the pressure to the mixture of the biomaterial scaffold and cells may comprise vacuum-sealing the mixture. In such embodiments, the mixture may be vacuum-sealed using any suitable technique. For example, in some embodiments, the mixture may be vacuum-sealed using a commercially available vacuum-sealer and compatible containers. In such embodiments, the mixture of the biomaterial scaffold and cells may be mixed, e.g. using two syringes connected by a luer lock, placed in the compatible container, and vacuum-sealed. Other envisioned mixing techniques may include stirring, beating, blending, cutting in, whisking, folding or emulsifying. Various types of equipment may also be utilized, including but not limited to, tumbler blenders, ribbon blenders, paddle mixers, agitators, emulsifiers, homogenizers, or heavy-duty mixers. As will be appreciated, such embodiments may be particularly useful if the seeded biomaterial scaffold is to be used in the food industry (e.g. to produce a cultured meat product), as the method may be performed, for example, in a kitchen using readily available kitchen-grade equipment.

In general, the pressure may be applied to the mixture of the biomaterial scaffold and cells for a period of time long enough to allow the cells to completely infiltrate the biomaterial scaffold. The amount of time required for the cells to infiltrate the biomaterial scaffold may depend in part on the type of cells, the composition of the biomaterial scaffold, and/or the preparation of the biomaterial scaffold. In some embodiments, the pressure may be applied to the mixture of the biomaterial scaffold and the cells for at least about 5 minutes. In a further embodiment, the pressure may be applied to the mixture for at least about 30 minutes. In some embodiments, the pressure may be applied for about up to about 7 days.

Further, in some embodiments, the mixture may be maintained under a selected temperature during application of the pressure. The temperature may be selected based on the intended application of the seeded biomaterial. For example, for food applications, it may be useful to maintain the mixture at temperatures that may preserve the biomaterial scaffold and cells during the application of pressure, particularly if the pressure is applied for an extended period of time (e.g. one or more days). In such embodiments, the mixture may be maintained at a temperature of about 0° C. to about 37° C., or about 4° C. to about 10° C. during the application of pressure. The mixture can also be treated with temperatures from about 30° C. to 100° C. For example, if a decrease in pressure is applied to the mixture, the mixture may be maintained under vacuum for a selected period of time within that temperature range. As will be appreciated, such a temperature range may be useful for food applications, as the temperature range is readily maintainable by consumer-grade refrigerators.

Thus, the methods of the present disclosure may produce a seeded, or fully-infiltrated, biomaterial scaffold in a considerably shorter period of time than the conventional processes. Further, the methods of the present disclosure also involve a minimal amount of steps to seed the biomaterial scaffold. As previously described herein, conventional processes are labour intensive and involve renewing cell culture media daily and re-plating the biomaterial scaffold about every 2 weeks for up to 12 weeks.

The methods of the present disclosure may also comprise crosslinking the mixture of the biomaterial scaffold and the cells. In some embodiments, the mixture is crosslinked prior to the application of pressure. In other embodiments, the mixture is crosslinked after the application of pressure. In such embodiments, the mixture may be removed from the positive or decrease in pressure and a crosslinker may be added thereto. In yet another embodiment, the mixture may be crosslinked simultaneously with the application of pressure thereto. For example, a crosslinker may be added to the mixture immediately prior to applying a pressure so that the crosslinking reaction proceeds during the application of the pressure.

In another embodiment, the biomaterial scaffold may be crosslinked prior to combining with the plurality of cells. In such embodiments, the biomaterial scaffold may be provided as a crosslinked biomaterial scaffold or, alternatively, the methods of the present disclosure may comprise crosslinking the biomaterial scaffold.

The crosslinking may be performed using any suitable crosslinker. As will be appreciated, the type of crosslinker used depends at least in part on the composition of the biomaterial scaffold. For example, if the biomaterial scaffold comprises an alginate, the mixture may be crosslinked using a crosslinker such as a CaCl₂ solution. As another example, if the biomaterial scaffold comprises a protein, such as a gelatin, the mixture may be crosslinked using a crosslinker such as a transglutaminase solution. Other protein types may include, without limitation: leguminous proteins (such as soy protein, mung bean protein, pea protein, kidney bean protein, lupin protein, and chickpea protein), cereal proteins (such as wheat protein, rice protein, and corn protein), oil seed proteins (such as peanut protein, sunflower protein, canola protein, flax seed protein, sesame protein), fungi protein (such as mycoprotein, yeast, mushrooms), and canola oil protein. As will be appreciated, the amount of crosslinker required may also depend on the composition of the biomaterial scaffold. In general, the crosslinker may be included in a suitable stock solution at a concentration of about 1% to about 10%. Of course, stock solutions of different concentrations may be used if so desired.

The biomaterial scaffold or the mixture of the biomaterial scaffold and the cells may be crosslinked for any suitable period of time. For example, in some embodiments, the biomaterial scaffold or the mixture may be crosslinked for at least about 30 minutes. In another embodiment, the biomaterial scaffold or the mixture may be crosslinked for about 30 minutes to about 24 hours. Of course, the biomaterial scaffold or the mixture may be crosslinked for less or more time as desired.

The methods of the present disclosure may comprise additional steps to prepare the seeded biomaterial scaffold for subsequent use. For example, as previously described herein, the methods of the present disclosure may be particularly well suited for food applications such as the production of cultured meat products.

In some embodiments, the methods of the present disclosure may further comprise directionally freezing the biomaterial scaffold and/or the mixture of the biomaterial scaffold and the cells. Directional freezing may provide the biomaterial scaffold with an aligned, porous structure similar to that of fibers found in natural meat products. The cells may then infiltrate and/or proliferate within the aligned porous structure of the biomaterial scaffold such that the resultant seeded biomaterial scaffold is fibrous similar to that of a natural meat product.

The directional freezing may be performed using any suitable technique, such as those described below in the Examples. In general, directional freezing involves creating a larger thermal gradient on one side of the biomaterial in order to form linear and highly aligned ice crystals extending from the cold side. This may force the components of the biomaterial scaffold to form around the ice crystals, thereby creating aligned microscale channels (pores). After directional freezing is performed, the ice crystals may be removed by, for example, sublimating the biomaterial scaffold.

In some embodiments, the formation of the aligned porous structure by way of directional freezing may be facilitated by the addition of one or more additivities capable of altering the structural properties of the aligned ice crystals such as sucrose, dextrose, trehalose, cornstarch, glycerol, ethanol, mannitol, sodium chloride, CaCl₂, gelatine, citric acid, dextran, sodium alginate, konjac gum/flour, locust bean, carrageenan, pectin, and the like.

Other techniques to mimic the structural and/or textural features of natural meat products may also be used instead of or in addition to directional freezing. For example, in some embodiments, the biomaterial scaffold or the seeded biomaterial scaffold may be divided into a plurality of strips that may subsequently be adhered together in a desired arrangement in order to imitate layers of a natural meat product. In such embodiments, the strips may be adhered together via crosslinking. In more detail, in some embodiments, the strips may be coated with a crosslinker and arranged into a desired formation to thereby provide a layered biomaterial scaffold. Such embodiments may be well suited for cultured fish products, as natural fish meat is generally layered.

Further, in order to facilitate the use of seeded scaffolds produced by the methods of the present disclosure in the production of cultured meat products, one or more colouring agents may be included in the biomaterial scaffold. Suitable colouring agents include natural colouring agents and artificial colouring agents. Examples of natural colouring agents include carotenoids, chlorophyllins, anthocyanins, annatto, caramel colouring, carmine, elderberry juice, lycopene, paprika, turmeric, beetroot powders, sweet potato powders, red iron oxidase, paprika oleoresin, titanium dioxide, calcium carbonate, super red and the like. Examples of artificial colouring agents include Allura Red, Amaranth, Erythrosine, Indigotine, Sunset Yellow FCF, Tartrazine, and the like. As will be appreciated, any combination of colouring agents may be included to provide the biomaterial scaffold or the seeded biomaterial scaffold with a desired colour.

Furthermore, in some embodiments, the biomaterial scaffold may be pre-treated to provide a desired flavor. For example, the biomaterial scaffold may be immersed in a broth prior to combining with the plurality of cells. The broth may be selected based on the meat product that the seeded biomaterial scaffold is intended to recreate. Non-limiting examples of suitable broths include vegan broths, beef broths, chicken broths, fish broths, or the like. The biomaterial scaffolds may be immersed in the broth for any suitable amount of time. As will be appreciated, the longer that the biomaterial scaffold is immersed, the more flavor is imparted thereto from the broth. In some embodiments, the biomaterial scaffold may be immersed for at least 20 minutes.

### Examples and Experimental Data

### Example 1: Studying the Penetration Capacity of C2C12 Cells into a Biomaterial Scaffold Using Cross-Linking and Vacuum Sealing Techniques

Four different seeded scaffolds were prepared using differing techniques. The seeded scaffolds were analyzed under microscope to examine any differences thereof in order to study how different preparation methods affect the resulting seeded scaffold.

Scaffold 1—Alginate/Mercerized Apple Scaffold, Crosslinked with CaCl₂ Before Seeding with 1.08×10⁷ C2C12 Cells

3 mL of a 5% sodium alginate solution, 4.5 mL of distilled water, and 7.5 g of mercerized apple (mercerized AA) were mixed and the mixture was used to produce a biomaterial scaffold using the process outlined in U.S. Provisional Application No. 63/107,226.

The scaffold was then lyophilized for 24 hours at −55° C. and 0.100 mbar. After lyophilization, the scaffold was cross-linked using a 0.1M CaCl₂ solution for 30 minutes. The resulting scaffold had a size of 60 mm by 15 mm.

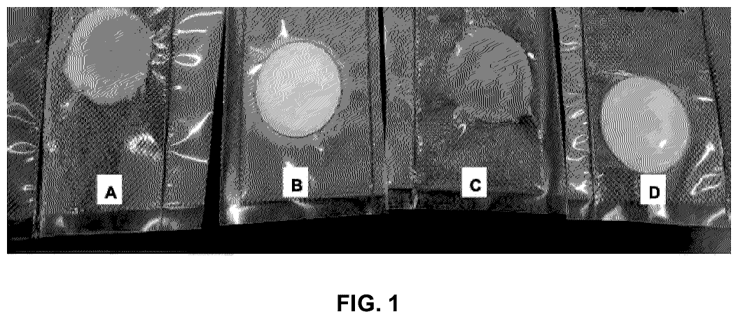

The scaffold was subsequently seeded with 1.08×10′ C2C12 cells and 5 mL of Dulbecco's Modified Eagle Medium (DMEM) containing 10% fetal bovine serum (FBS) and 1% Penicillin/Streptomycin as a cell culture media. The seeded scaffold was then vacuum-sealed in a plastic enclosure using a commercially available vacuum sealer, as shown in FIG. 1D. The vacuum-sealed scaffold was then maintained in a fridge for 7 days.

After 7 days, the scaffold was removed from the plastic enclosure and washed three times with phosphate-buffered saline (PBS). The washed scaffold was then fixed with a 4% paraformaldehyde solution (PFA) in PBS for 1 hour. After fixing, the scaffold was subsequently washed a further three times with PBS and three samples were dissected therefrom.

The samples were each stained with Hoechst for 30 minutes and then washed a further three times with PBS.

Scaffold 2—Alginate/Mercerized AA Scaffold, Crosslinked with CaCl₂ after Seeding with 2.81×10⁷ C2C12 Cells

3 mL of a 5% sodium alginate solution, 4.5 mL of distilled water, and 7.5 g of mercerized AA were mixed and the mixture was used to produce a biomaterial scaffold using the process outlined in U.S. Provisional Application No. 63/107,226.

The scaffold was then lyophilized for 24 hours at −55° C. and 0.100 mbar. The resulting scaffold had a size of 60 mm by 15 mm. After lyophilization, the scaffold was seeded with 2.81×10⁷ C2C12 cells and 5 mL of DMEM containing 10% FBS and 1% Penicillin/Streptomycin as a cell culture media.

After 15 minutes, the seeded scaffold was cross-linked using a 0.1M CaCl₂ solution for 30 minutes. The seeded scaffold was then vacuum-sealed in a plastic enclosure using a commercially available vacuum sealer, as shown in FIG. 1C. The vacuum-sealed scaffold was then maintained in a fridge for 6 days.

After the 6 days, the scaffold was removed from the plastic enclosure and washed three times with PBS. The washed scaffold was then fixed with a 4% PFA in PBS for 1 hour. The fixed scaffold was subsequently washed a further three times with PBS and three samples were dissected therefrom.

The samples were each stained with Hoechst for 30 minutes and then washed a further three times with PBS.

Scaffold 3—Gelatin/Mercerized AA Scaffold, Crosslinked with Transglutaminase Before Seeding with 2.81×10⁷ C2C12 Cells

3 mL of a 20% fish gelatin solution, 4.5 mL of distilled water, and 7.5 g of mercerized AA were mixed and the resulting mixture was used to produce a biomaterial scaffold using the process outlined in U.S. Provisional Application No. 63/107,226.

The scaffold was then lyophilized for 24 hours at −55° C. and 0.100 mbar. After lyophilization, the scaffold was cross-linked using a 1% transglutaminease solution for 24 hours in a petri dish. The resulting scaffold had a size of 60 mm by 15 mm.

The crosslinked scaffold was subsequently seeded with 2.81×10′ C2C12 cells and 5 mL of DMEM containing 10% fetal bovine serum FBS and 1% Penicillin/Streptomycin as a cell culture media. The seeded scaffold was crosslinked again using the 1% transglutaminease solution for 24 hours. After the second crosslinking, the seeded scaffold was vacuum-sealed in a plastic enclosure using a commercially available vacuum sealer, as shown in FIG. 1B. The vacuum-sealed scaffold was then maintained in a fridge for 6 days.

After the 6 days, the scaffold was removed from the plastic enclosure and washed three times with PBS. The washed scaffold was then fixed with a 4% PFA in PBS for 1 hour. After fixing, the scaffold was washed a further three times with PBS and three samples were dissected therefrom.

The samples were each stained with Hoechst for 30 minutes and then washed a further three times with PBS.

Scaffold 4—Gelatin/Mercerized AA Scaffold, Crosslinked with Transglutaminase and Seeding with 1.08×10⁷ C2C12 Cells

3 mL of a 20% fish gelatin solution, 4.5 mL of distilled water, and 7.5 g of mercerized AA were mixed and the resulting mixture was used to produce a biomaterial scaffold using the process outlined in U.S. Provisional Application No. 63/107,226.

The scaffold was then lyophilized for 24 hours at −55° C. and 0.100 mbar. The resulting scaffold had a size of 60 mm by 15 mm. After lyophilization, the scaffold was seeded with 2.08×10′ C2C12 cells and 5 mL of DMEM containing 10% fetal bovine serum FBS, 1% Penicillin/Streptomycin, and 10% transglutaminase as a cell culture media. The seeded scaffold was left to crosslink in a petri dish for 24 hours.

After seeding and crosslinking, the scaffold was vacuum-sealed in a plastic enclosure using a commercially available vacuum sealer. The vacuum-sealed scaffold was then maintained in a fridge for 6 days.

After the 6 days, the scaffold was removed from the plastic enclosure and washed three times with PBS, as shown in FIG. 1A. The washed scaffold was then fixed with a 4% PFA in PBS for 1 hour. After fixing, the scaffold was washed a further three times with PBS and three samples were dissected therefrom.

The samples were each stained with Hoechst for 30 minutes and then washed a further three times with PBS.

### Results

Each of the samples of the Scaffolds 1-4 were analyzed under microscope and pictures were taken, as outlined below.

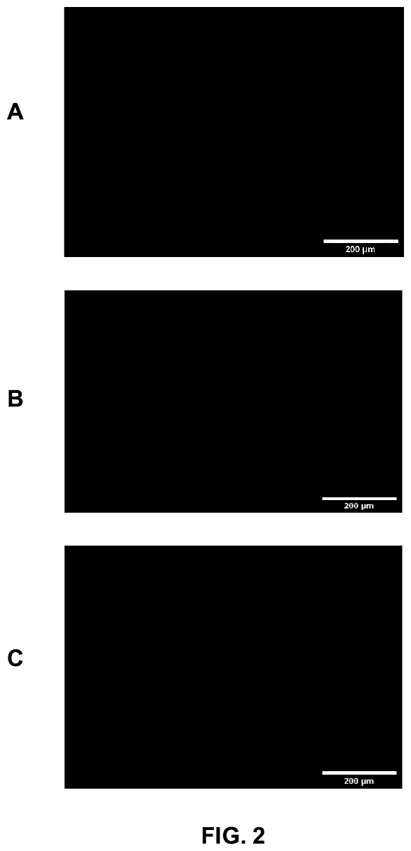

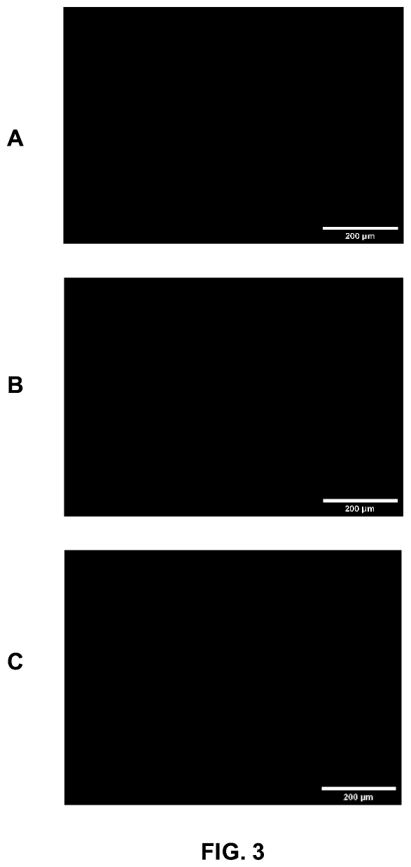

FIG. 2A, FIG. 2B, and FIG. 2C show a microscopy image of a top side, a bottom side, and an interior of one of the samples of Scaffold 1, respectively, while FIG. 3A, FIG. 3B, and FIG. 3C show a top side, a bottom side, and an interior of one of the samples of Scaffold 2, respectively.

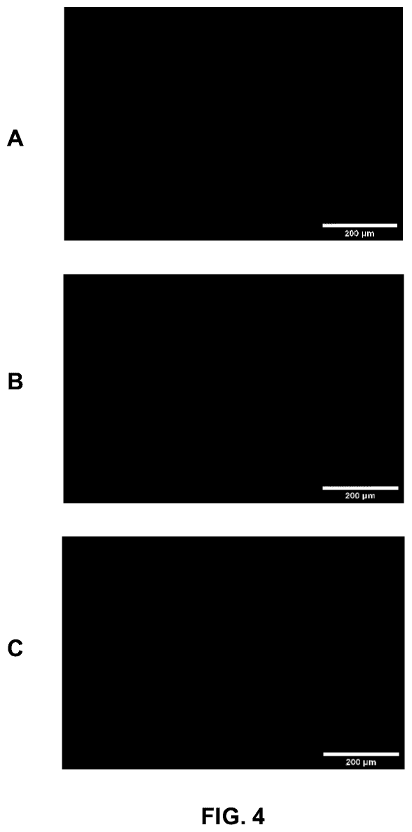

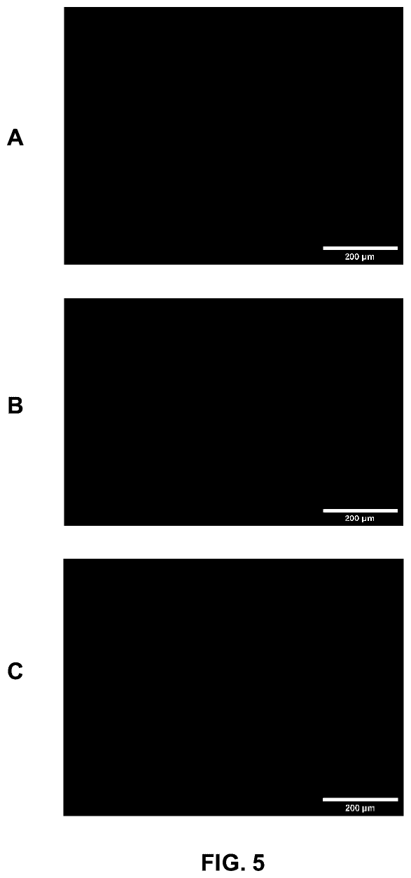

FIG. 4A, FIG. 4B, and FIG. 4C show a microscopy image of a top side, a bottom side, and an interior of one of the samples of Scaffold 3, respectively, while FIG. 5A, FIG. 5B, and FIG. 5C show a top side, a bottom side, and an interior of one of the samples of Scaffold 4, respectively.

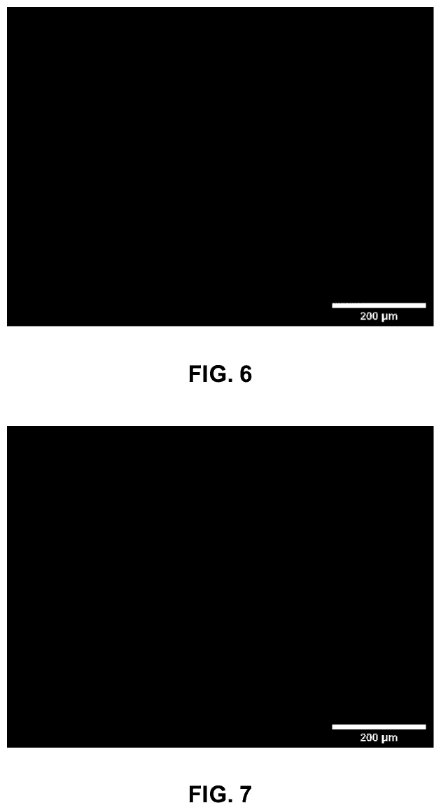

FIG. 6 shows a microscopy image of a longitudinal section of one of the samples of Scaffold 1, while FIG. 7 shows a microscopy image of a longitudinal section of one of the samples of Scaffold 3.

### Example 2: Comparison Between Cell Count Known Meat Standards and Biomaterial Scaffolds Seeded Using Cross-Linking and Vacuum Sealing Techniques

Three different seeded biomaterial scaffolds were prepared using differing techniques and analyzed under microscope to compare the cell counts thereof to known meat standards.

### Scaffold 5—Lyophilized Puck

A scaffold was prepared as described above using a mixture of 2% sodium alginate solution and mercerized AA at a ratio of 1:1. The scaffold was lyophilized for 48 hours at −55° C. and 0.100 mbar.

After lyophilization, the scaffold was cut with a 1 cm knife and subsequently seeded with 500 μL of 7×10⁸ GFP 3T3 cells (10 cell plates with confluency 100%) in DMEM containing 10% FBS and 1% Penicillin/Streptomycin as the cell culture media. Once seeded, the scaffold was cross-linked using 200 μL of a 1% CaCl₂) solution for 30 minutes.

The crosslinked scaffold was subsequently vacuum-sealed in a plastic enclosure using a commercially available vacuum sealer. The vacuum-sealed scaffold was then maintained in a fridge for 24 hours. After the 24 hours, the scaffold was washed three times with PBS, and then fixed with a 4% PFA in PBS for 1 hour.

The fixed scaffold was then washed a further three times with PBS and crosslinked again with a 1% CaCl₂) solution for 1 hour. After the additional crosslinking, the scaffold was stained with Hoechst for 30 minutes and washed a further three times with PBS.

The stained scaffold was analyzed using a microscope and a cell count was performed thereon.

### Scaffold 6—“Mix in Syringe” Puck

A 1 mL mixture of 2% sodium alginate solution, mercerized AA, and 200 μL of 3.10×10¹ GFP 3T3 cells (10 cell plates with confluency 100%) in DMEM containing 10% FBS and 1% Penicillin/Streptomycin as a cell culture media was prepared using locked syringes.

The mixture was homogenized 20 times inside a syringe to produce a scaffold, which was then transferred to a petri dish. The scaffold was subsequently crosslinked using 200 μL of a 1% CaCl₂) for 30 min. The crosslinked scaffold was then vacuum-sealed in a plastic enclosure using a commercially available vacuum sealer. The vacuum-sealed scaffold was maintained in a fridge for 24 hours.

After the 24 hours, the scaffold was washed three times with PBS and then fixed with a 4% PFA in PBS for 1 hour. After fixing, the scaffold was washed a further three times with PBS and crosslinked again with a 1% CaCl₂) solution for 1 hour. After the additional crosslinking, the scaffold was stained with Hoechst for 30 minutes and washed a further three times with PBS.

The stained scaffold was analyzed using a microscope and a cell count was performed thereon.

### Scaffold 7—Negative Control

A scaffold was prepared as described above using a mixture of 2% sodium alginate solution and mercerized AA at a ratio of 1:1. The scaffold was lyophilized for 48 hours at −55° C. and 0.100 mbar.

After lyophilization, the scaffold was cut with a 1 cm knife and subsequently seeded with 500 μL of distilled water. Once seeded, the scaffold was cross-linked using 200 μL of a 1% CaCl₂) solution for 30 minutes.

The crosslinked scaffold was subsequently vacuum sealed in a plastic enclosure using a commercially available vacuum sealer. The vacuum sealed scaffold was then maintained in a fridge for 24 hours. After the 24 hours, the scaffold was washed three times with PBS, and then fixed with a 4% PFA in PBS for 1 hour.

The fixed scaffold was then washed a further three times with PBS and crosslinked again with a 1% CaCl₂) solution for 1 hour. After the additional crosslinking, the scaffold was stained with Hoechst for 30 minutes and washed a further three times with PBS.

The stained scaffold was then analyzed using a microscope.

### Meat Standards

Scallop, lean beef (interior round muscle), and tuna samples were prepared by cutting each of the meats parallel to the fibers thereof and perpendicular to the fibers thereof.

Each of the samples were fixed with a 4% PFA in PBS for 24 hours and then washed three times with PBS. The washed samples were then each stained again with Hoechst for 30 min and washed a further three times with PBS.

The samples were subsequently analyzed using a microscope and a cell count was performed thereon.

### Results

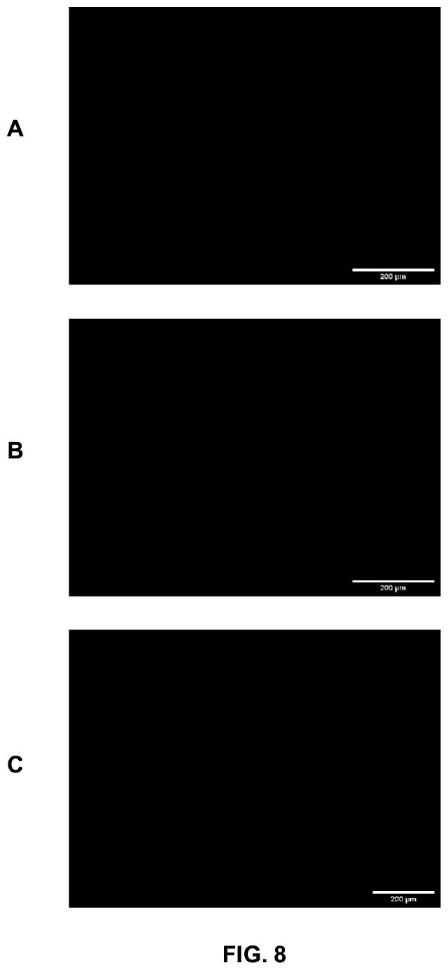

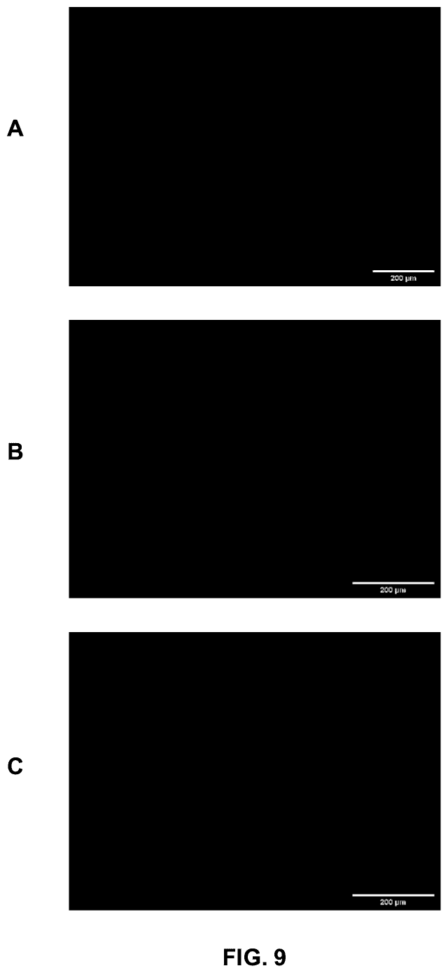

Microscopy images of the stained samples were taken are shown in FIG. 8 and FIG. 9. FIG. 8A shows a microscopy image of Scaffold 5, FIG. 8B shows a microscopy image of Scaffold 6, and FIG. 8C shows a microscopy image of the Negative Control. FIG. 9A shows a microscopy image of one of the tuna samples, FIG. 9B shows a microscopy image of one of the scallop samples, and FIG. 9C shows a microscopy image of one of the beef samples.

### Example 3: Studying the Penetration Capacity of C2C12 Cells into a Biomaterial Scaffold Using Mixing, Cross-Linking, and Vacuum Techniques

C2C12 myoblast cells were grown in 20 cell plates until a confluency of 100% was achieved. The cells were then resuspended in 200 μL of DMEM containing 10% FBS and 1% Penicillin/Streptomycin as a cell culture media.

A 2 mL mixture of 2% sodium alginate solution (final concentration of 1%; 1 mL), centrifuged mercerized AA (centrifuged; 1 mL), and 200 μL of 5.5×107 of the resuspended C2C12 myoblast cells was prepared using locked syringes.

The mixture was subsequently homogenized 20 times between the locked syringes to form a scaffold. The scaffold was then crosslinked using a 1% CaCl₂) dihydrate solution for 1 hour. The scaffold was subsequently flipped and maintained in the 1% CaCl₂) dihydrate solution for another hour. After, the scaffold was vacuum-sealed and maintained in a fridge for 7 days.

After the 7 days, the scaffold was washed three times and then fixed with a 4% PFA in PBS for 1 hour. The fixed scaffold was then washed a further three times with PBS. After washing, the scaffold was stained with Hoechst (1:1000, Hoechst:PBS) for 30 min and washed a further three times with PBS.

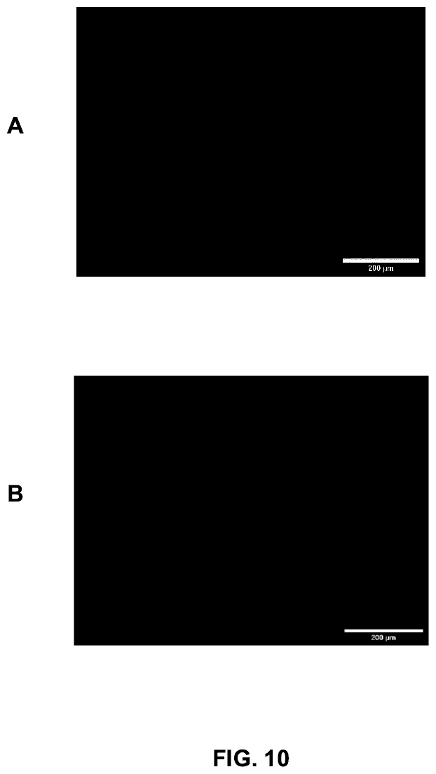

The stained scaffold was analyzed under a microscope having a DAPI filter applied. Images were taken and are shown in FIGS. 10A and 10B, which respectively show an external view of the scaffold and an internal view of the scaffold.

The stained scaffold was then further stained with a 0.01% Congo red solution for 30 minutes and washed a further three times with PBS for 30 minutes.

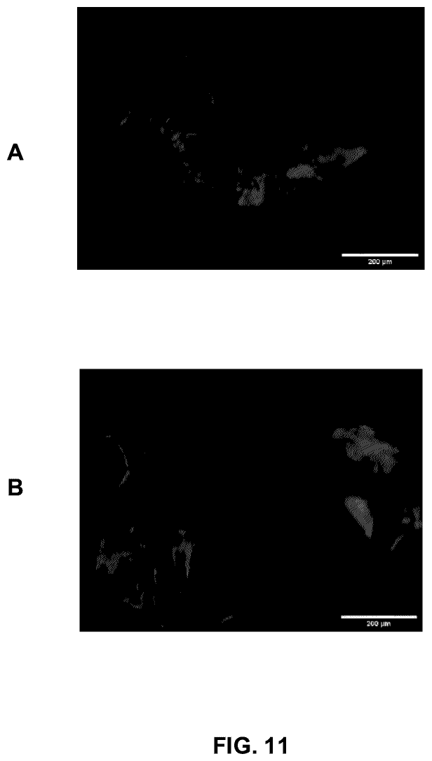

The further stained scaffold was analyzed under a microscope having a TXRED filter applied. Images were taken and are shown in FIGS. 11A and 11B, which respectively show an external view of the scaffold and an internal view of the scaffold.

### Example 4: Studying the Dispersion Capacity of C2C12 Cells into a Biomaterial Scaffold Using Mixing, Cross-Linking, and Vacuum Techniques

C2C12 myoblast cells were grown in 30 cell plates until a confluency of 100% was achieved. The cells were then resuspended in 1 mL of DMEM containing 10% FBS and 1% Penicillin/Streptomycin as a cell culture media.

A 5 mL mixture of 2% sodium alginate solution (final concentration of 1%; 2.5 mL), centrifuged mercerized palm heart (centrifuged; 2.5 mL), and 400 μL of 1.17×10⁸ of the resuspended C2C12 myoblast cells was prepared using locked syringes.

The mixture was subsequently homogenized 20 times between the locked syringes to form a scaffold. The scaffold was then crosslinked using a 1% CaCl₂) dihydrate solution for 1 hour in a fridge. After, the scaffold was vacuum-sealed using a commercially available vacuum sealer and maintained in a fridge for 2 days.

After the 2 days, the scaffold was washed three times and the fixed with a 4% PFA in PBS for 1 hour. The fixed scaffold was then washed a further three times with PBS. After washing, the scaffold was stained with Hoechst (1:1000, Hoechst:PBS) for 30 min and washed a further three times with PBS. The stained scaffold was analyzed under a microscope having a DAPI filter applied.

The stained scaffold was then further stained with a 0.01% Congo red solution for 30 minutes and washed a further three times with PBS for 30 minutes. The further stained scaffold was analyzed under a microscope having a TXRED filter applied.

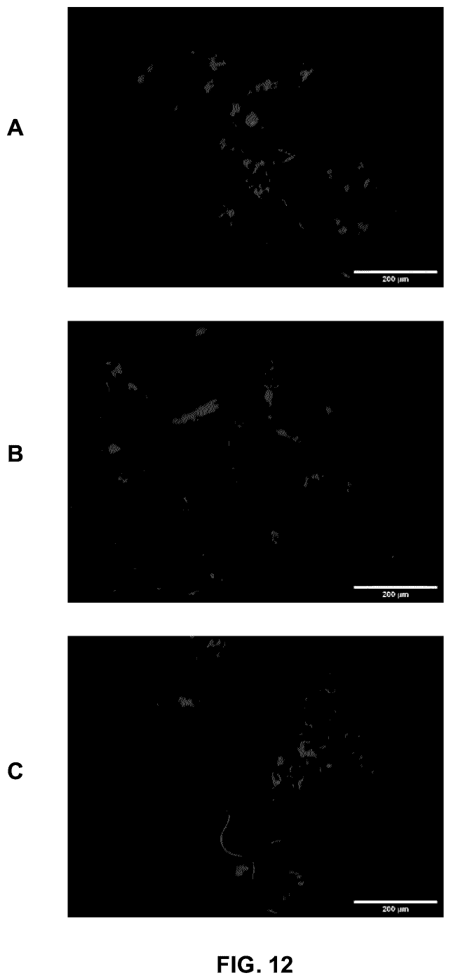

A composite of images taken under the DAPI filter and under the TXRED filter were created and are shown in FIG. 12, wherein FIG. 12A is a microscopy image of a textured edge of the stained scaffold, FIG. 12B is a microscopy image of an interior of the stained scaffold, and FIG. 12C is a microscopy image of a smooth edge of the stained scaffold.

### Example 5: Comparison Between Cell Counts of Meat Samples and Seeded Biomaterial Scaffolds Produced by the Methods Described Herein

Cell counts were performed on the seeded scaffold produced in Examples 3 and 4. The cell counts of the scaffolds were compared to the cell counts of different types of meat samples. The results are shown below in Table 1.

TABLE 1
Cell counts of scaffolds produced by the methods
of the present disclosure and meat samples.

Cell Counts

| Sample | Average | Standard Deviation |
|---|---|---|
| Scallop | 1163 | 142 |
| Tuna | 1001 | 304 |
| Beef | 442 | 133 |
| Scaffold (Method 3) | 1083 | 144 |
| Scaffold (Method 4) | 943 | 438 |

As can be seen from Table 1, the methods described herein are capable of producing seeded scaffolds having cell counts similar to those of meat samples and thus may be particularly useful for producing meat products by way of 3D scaffolding.

### Example 6: Investigating Kitchen-Safe Mercerization Processes

As previously described herein, in some embodiments, the methods of the present disclosure may be “food grade” such that they may be performed in kitchens. In some cases, it may be desired to mercerize ingredients in a kitchen or other non-lab setting prior to use in the methods of the present disclosure. Standard mercerization procedures involve the use of 1 M NaOH and are not suitable to be performed in, for example, a shared kitchen. Thus, alternative mercerization techniques were investigated and are discussed below.

2.5 L of a 10% sodium bicarbonate solution having an initial pH of 7.84 was added to a 4 L beaker and heated to 80° C. 250 g of decellularized apple was then added to the heated solution and mixed therewith.

25 mL of 30% hydrogen peroxide stock solution was added to the heated solution with stirring at 15 minute intervals to a total of 125 mL. The stirring was performed for about 1 hour in total. The solution was then removed from the heat source. The solution had a pH of 8.65. Glacial acetic acid was then added to the solution until the solution had a pH of 7.10.

A high degree of carbonation resulted from the reaction of the sodium bicarbonate and the acetic acid. As a result, the solution was agitated by shaking and pouring between beakers to thereby minimize the chance of a pressure buildup in subsequent steps. After agitation, 25 mL aliquots of the solution were added to 50 mL falcon tubes and centrifuged at 5000 rpm for 5 minutes. The tubes were then examined to see if any residual pressure buildup was present in the tubes. No bubbling or gas release occurred.

The samples were then transferred to 1 L tubes and centrifuged for 15 minutes at 8000 rpm. After 5 minutes of centrifuging, the tubes were examined for any pressure buildup (e.g. bubbling or other gas release). After centrifuging, the supernatant liquid was discarded from each of the tubes and replaced with distilled water. This process was repeated four times as outlined below.

After the first centrifugation, the pH of samples was 8.76. The samples were then neutralized to a pH of 6.82. After the second centrifugation, the pH of the resulting samples was 7.70. The resulting samples were subsequently neutralized to a pH of 6.91. After the third centrifugation, the resulting samples had a pH of 6.88. No neutralization step was required at this pint and the samples were centrifuged once more and then stored in a 50 mL falcon tube.

The yield of mercerized apple was 55.30 g.

### Example 7: Investigating Further Kitchen-Safe Mercerization Processes

Another process for mercerization of ingredients to be used in the methods of the present disclosure is outlined below. Again, the process was designed to be “food grade” such that it may be performed in, for example, a kitchen rather than a lab.

2.5 L of a 10% sodium bicarbonate solution was added to a 4 L beaker containing therein 277.4 g of decellularized apple to form a mixture. The solution and the decellularized apple were mixed at room temperature for 5 days. The pH of the solution was monitored over the course of the 5 days: before mixing the pH was 7.94, on day 1 the pH was 8.25, on day 2 the pH was 8.54, and on day 5 the pH was 8.98.

After the 5 days of mixing, 25 mL of 30% hydrogen peroxide stock solution was added at 15 minute intervals until a total of 125 mL was added. No bleaching was observed after adding the hydrogen peroxide, so the temperature of the solution was increased to 80° C. over the course of 1 hour.

The solution was then neutralized by adding glacial acetic acid until the solution had a pH of 7.10. After neutralizing the solution, the solution was agitated by shaking and pouring between beakers in order to reduce the chances of pressure buildup during subsequent centrifugation.

After agitation, the solution was separated into 1 L tubes and centrifuged at 8000 rpm for 15 minutes. After centrifuging, the supernatant was discarded and distilled water was added. This process was repeated eight times. The pH values recorded are outlined below:
pH of 7.10 after the first neutralization, before centrifugation
pH of 8.62 after the first centrifugation, neutralized to 7.10
pH of 7.63 after the second centrifugation, neutralized to 7.17
pH of 7.85 after the third centrifugation, neutralized to 7.12
pH of 7.69 after the fourth centrifugation, neutralized to 6.87
pH of 7.29 after the fifth centrifugation, neutralized to 6.87
pH of 7.53 after the sixth centrifugation, neutralized to 6.77
pH of 7.10 after the seventh centrifugation

After the seventh centrifugation, the samples were centrifuged once more and then stored in a 50 mL falcon tube.

The yield of mercerized apple was 63.9 g.

### Example 8: Comparison Between Standard and Food Grade Mercerization Processes

The food grade mercerization processes of Examples 6 and 7 were compared to a standard mercerization process. The standard process was identical to that exemplified in Example 6 except that 1M NaOH was used instead of the 10% sodium bicarbonate solution.

4.5 g of samples from each of the processes were each mixed with 3 mL of a 5% sodium alginate solution and 4.5 mL of distilled water. Each mixture was frozen and then freeze-dried for 48 hours.

Each of the samples was then analyzed using microscopy and Fourier-transform infrared spectroscopy (FTIR).

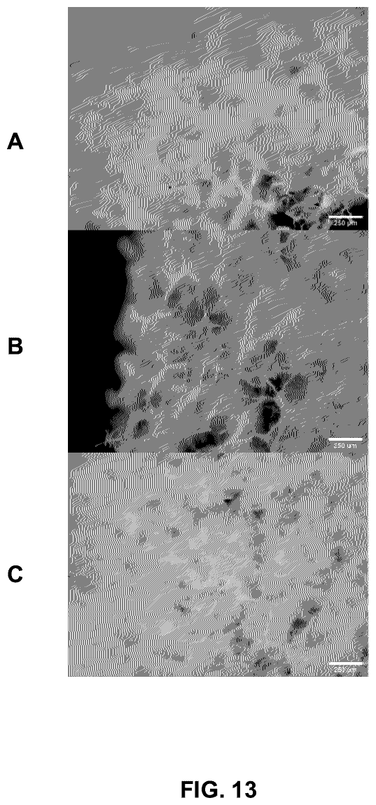

For microscopy, the samples were visualized in the dark field using 1× and 6.3× magnifications. The results are shown in FIG. 13, wherein FIG. 13A is a microscopy image of a sample produced by the process of Example 7, FIG. 13B is a microscopy image of a sample produced by the process of Example 6, and FIG. 13C is a microscopy image of a sample produced by the standard process. As can be seen, the samples produced by the processes exemplified in Example 6 and Example 7 are microscopically similar to those produced using an equivalent standard process.

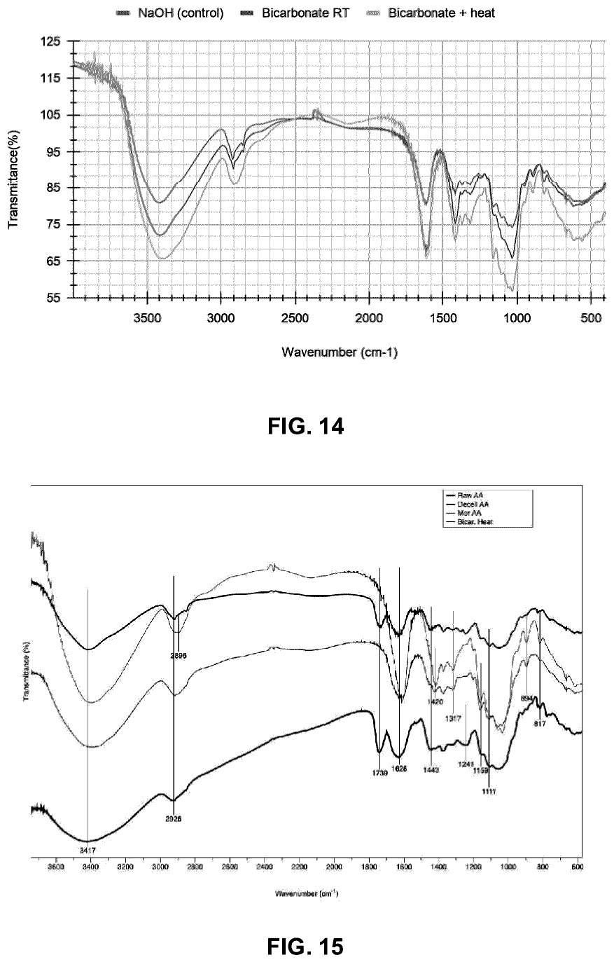

For FTIR analysis, a potassium bromide control was prepared and dried in an oven for 24 hours. The potassium bromide control was analyzed and used to reduce background noise for the FTIR analysis of the samples. The samples were analyzed using the following FTIR settings: Start: 4000.0 and End: 400.0; Scan: 32; Resolution: 2. The results are shown in FIG. 14. As another comparison, the FTIR results of the samples produced from the process of Example 6 and the standard process were compared to those of decellularized apple, and raw apple, as shown in FIG. 15.

The FTIR analysis showed similar trends for each of the different samples, indicating that there are similarities in the functional groups present. This means that bicarbonate mercerization could be used as a viable kitchen-safe replacement to NaOH mercerization.

In more detail, in the FTIR analysis, the peaks in the range 3600 cm⁻¹ to 2925 cm⁻¹ are associated with free O—H stretching vibration of the OH groups in cellulose molecules and hydrogen bonded OH stretching vibrations. The range 2925 cm⁻¹ to 2880 cm⁻¹ corresponds to aliphatic saturated C—H stretching associated with methylene groups in cellulose.

Lignin may also be assigned a broad region including an interval 3300 cm⁻¹ to 3600 cm⁻¹ (intramolecular hydrogen bond in phenolic groups, OH stretching of alcohols, phenols, acids and weakly bounded absorbed water). Lignin is composed of three basic units, namely p-hydroxyphenyl (H), guaiacyl (G) and syringyl (S). Guaiacyl (G) and syringyl (S) are the main units of lignin, but the ratio of S/G varies from one to another plant. The bands at the 1241 cm⁻¹ and 1317 cm⁻¹ may be assigned as G-ring stretching and S ring stretching respectively. The presence of the band at 1241 cm⁻¹, which may be assigned to C—O stretching vibration in xyloglucan, in only raw and decellularized cellulose indicates that bicarbonate is effective at removing lignin.

Further, the FTIR spectra including an interval between 1750 cm⁻¹ and 1700 cm⁻¹, which may be assigned to C═O stretching in unconjugated groups, reflects changes in various functional groups in lignin and hemicelluloses such as carbonyls, ester groups, ketones, aldehydes, and carboxylic acids. The absence of the band at 1740 cm⁻¹ for the samples mercerized with bicarbonate also indicates that bicarbonate is effective at removing lignin and hemicellulose from the raw and decellularized material.

In addition to the above analyses, a sample of mercerized apple produced by each of the processes was analyzed to determine the Feret diameter of the particles thereof. To determine the Feret diameters, a 0.5 g sample of mercerized apple produced by each of the processes was mixed with 0.5 mL of a 0.2% Congo red solution. The mixture was diluted by adding 1 mL thereof to 7 mL of distilled water. Then, 1 mL of the diluted mixture was added to another 7 mL of distilled water for further dilution. A few drops of the further diluted mixture was added to a glass slide and covered with a cover slip to be analyzed under a TXRED filter.

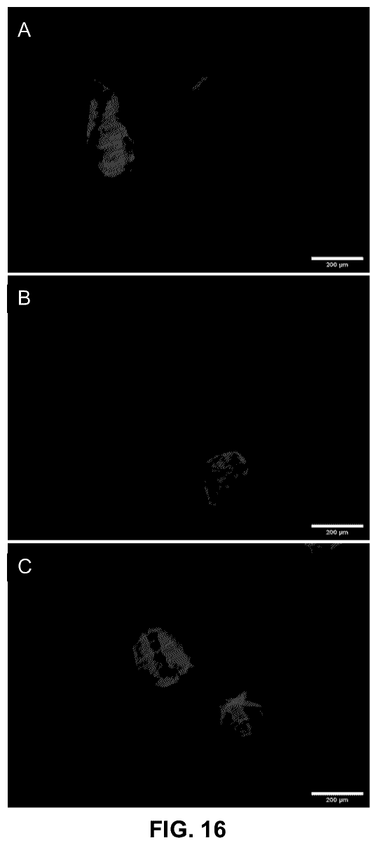

Referring now to FIG. 16, FIG. 16A shows single particles of a sample produced by the process of Example 7, FIG. 16B shows a single particle of a sample produced by the process of Example 6, and FIG. 16C shows single particles of a sample produced using the standard process. The average sizes of the particles for each sample are shown in Table 2.

TABLE 2
Average Feret diameters of sample particles produced by the
processes of Example 6, Example 7, and the standard process.

| | Average Feret | Standard |
|---|---|---|
| Mercerization Method | diameter (μm) | Deviation (μm) |
| Standard Process | 227.0 | 82.8 |
| Example 6 Process | 300.6 | 89.7 |
| Example 7 Process | 278.2 | 94.8 |

SD—standard deviation; Xlstat 2014
^{A-B}Means in a column without common superscripts are different (P < 0.05).

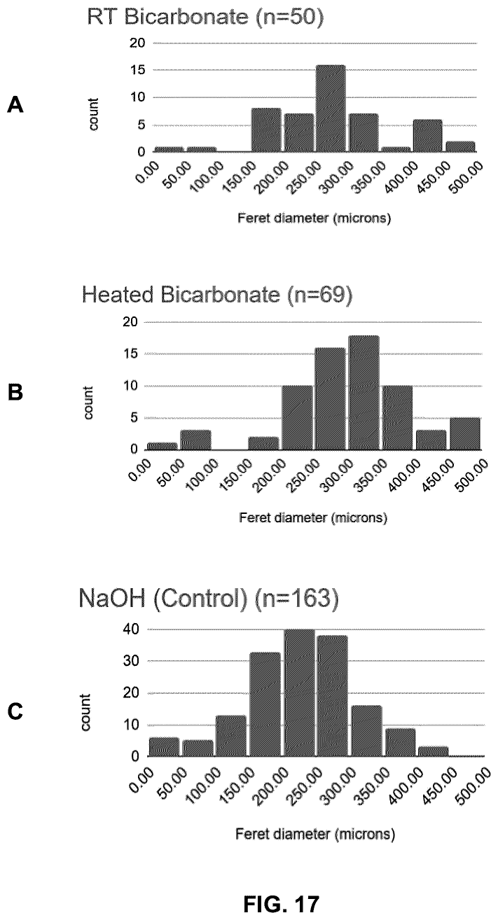

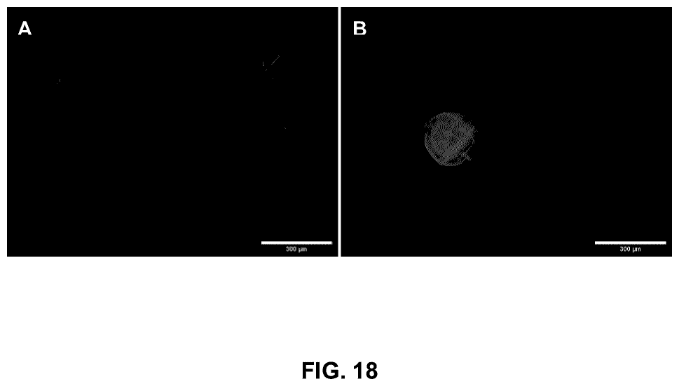

The particles of each sample analyzed were also counted. The results are shown in FIG. 17, wherein FIG. 17A is a histogram illustrating the number of particles in specific size ranges for the sample produced by the process of Example 7, FIG. 17B is a histogram illustrating the number of particles in specific size ranges for the sample produced by the process of Example 6, and FIG. 17C is a histogram illustrating the number of particles in specific size ranges for the sample produced by the standard process.

In general, the Feret diameter of sample particles formed by the standard process were smaller than those produced by the processes of Example 6 and Example 7. In addition, the sample particles produced by the processes of Example 6 and Example 7 did not demonstrate a significant difference in Feret diameter with each other (P>0.05). Further, as shown in FIG. 17, particles of the samples produced by standard process demonstrated a normal distribution, whereas the particles of the samples produced by the processes of Example 6 and Example 7 exhibited a slight left skew. For all three processes, every particle was under 500 μm.

Thus, it is clear that sodium bicarbonate is an effective alternative to NaOH for mercerization. As sodium bicarbonate is generally regarded as safe, such mercerizations may be performed outside of a lab (e.g. in a kitchen).

### Example 9: Studying the Effect of Hydrogen Peroxide Stock Solution Concentration During the Mercerization

As previously described herein, conventional mercerization processes used to produce biomaterial scaffolds may involve bleaching the biomaterials with a hydrogen peroxide solution. Typically, the hydrogen peroxide solution is a 30% hydrogen peroxide stock solution. However, as will be appreciated, such concentrations may not be suitable for use in a kitchen environment—i.e. such solutions may not be kitchen-safe.

Thus, this study sought to investigate the suitability of lower-concentration hydrogen peroxide stock solutions for bleaching biomaterials during mercerization.

Nine McIntosh apples (955 g) were inspected, washed, peeled, and then de-cored using a commercially available corer. The apples were subsequently cut in quarters and ground using a food processor. The ground apple (700 g) was added to a 4 L beaker.

The ground apple was then decellularized using a 0.1% sodium dodecyl sulfate (SDS) solution and a commercially available stand mixer (130 rpm). The decellularized apple was then mercerized using a 10% sodium bicarbonate and 15% hydrogen peroxide stock solution with 1 hour of heating. The mercerized material was neutralized using a 25 μm sieve and glacial acetic acid. The mercerized material was passed through the sieve until a pH in the range of 6.8-7.2 was obtained.

The mercerized material was then prepped for particle imaging and FTIR analyses.

For particle imaging, a mix of 0.5 mL Congo Red (0.2% solution) with 0.5 g mercerized material was first prepared. The mixture was then diluted by adding 1 mL of the Tube 1 to 7 mL of dH₂O. The diluted mixture was further diluted by adding 1 mL of the diluted mixture to a further 7 mL of dH₂O. A few drops of the final diluted mixture were added to a glass slide and covered with a cover slip at which point they were imaged under microscope with a TXRED fluorescent filter applied. Using ImageJ, the image was treated, red was added, and the ferret diameters of the particles were measured.

For FTIR, a potassium bromide sample was prepared and left in an oven for at least 24 hours before being formed into a tablet. The potassium bromide table was analyzed and used to eliminate background noise for subsequent readings of the mercerized sample. The mercerized samples were analyzed using range: 4000.0 (start) and 400.0 (end); scan: 32; and resolution: 2.

As a comparison, samples prepared the same way except mercerized using a 10% bicarbonate and 30% hydrogen peroxide stock solution or a 1M NaOH and 30% hydrogen peroxide stock solution were also analyzed.

### Results

Microscopy images are from the samples mercerized with 10% sodium bicarbonate and 15% hydrogen peroxide or 30% hydrogen peroxide stock solutions are shown in FIG. 18A and FIG. 18B, respectively.

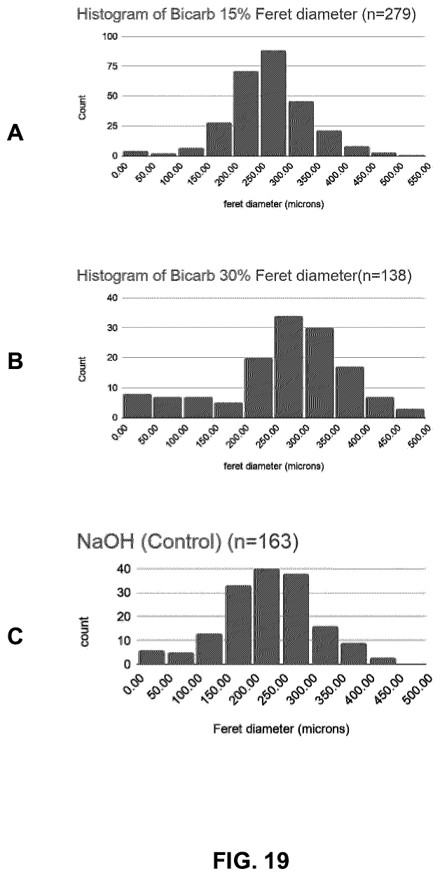

The particle size distribution of each of the samples is shown in FIG. 19, wherein FIG. 19A shows the particle distribution of the sample prepared using the 10% sodium bicarbonate and 15% hydrogen peroxide stock solution, FIG. 19B shows the particle distribution of the sample prepared using the 10% sodium bicarbonate and 30% hydrogen peroxide stock solution, and FIG. 19C shows the particle distribution of the sample prepared using the 1M NaOH and 30% hydrogen peroxide stock solution. The average Feret diameter of each of the samples is shown below in Table 3.

TABLE 3
Average Feret diameters of samples produced using
different concentrations of hydrogen peroxide.

| | Average Feret | SD Feret |
|---|---|---|
| Mercerization/Bleaching Method | Diameter (μm) | Diameter(μm) |
| 1M NaOH, 30% H₂O₂ | 227.0^{B} | 82.8 |
| 10% Sodium Bicarbonate, 30% H₂O₂ | 267.9^{A} | 105.6 |
| 10% Sodium Bicarbonate, 15% H₂O₂ | 266.9^{A} | 76.3 |

SD—standard deviation; Xlstat 2014
^{A-B}Means in a column without common superscripts are different (P < 0.05).

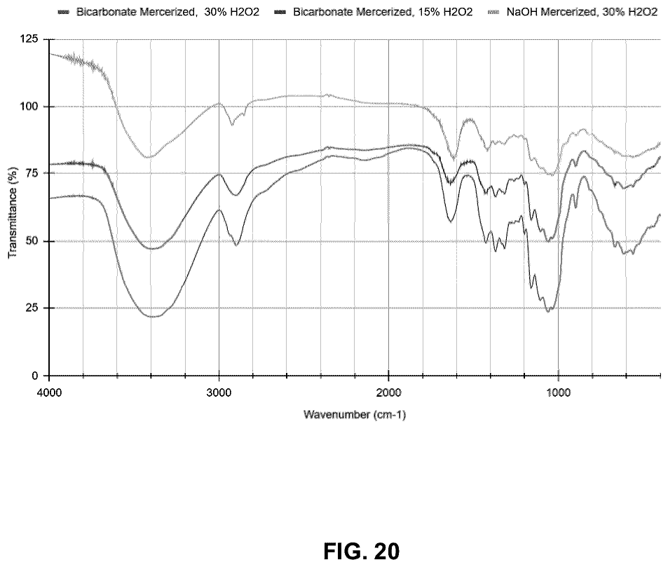

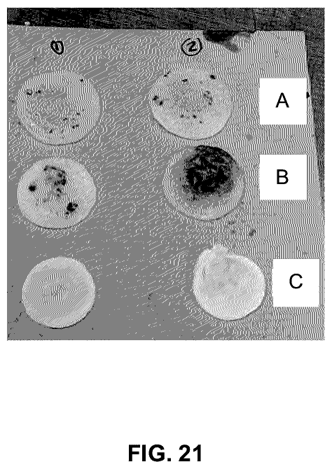

The FTIR results are shown in FIG. 20, wherein the blue line is the 10% sodium bicarbonate, 30% H₂O₂ stock solution sample, the red line is the 10% sodium bicarbonate, 15% H₂O₂ stock solution sample, and the orange line is the 1M NaOH, 30% H₂O₂ stock solution sample.

As shown from the above results, while the average Feret diameters of the 1M NaOH, 30% H₂O₂ stock solution sample is generally smaller than the other samples, the Feret diameters of the sodium bicarbonate samples were not significantly different (P>0.05). Further, as shown by the FTIR analysis, the chemical structure of each of the samples were similar.

Thus, the use of a lower-concentration of hydrogen peroxide stock solution for bleaching during the mercerization process did not result in any major differences and, as a result, represents a viable option for performing the methods of present disclosure under entirely kitchen-safe conditions.

### Example 10: Production of Food Grade Biomaterial Scaffolds in Kitchen Environment

As described above, the methods of the present disclosure may in some embodiments be kitchen-safe in that the reagents, experimental conditions, and/or equipment used may be suitable for handling or performing in a kitchen rather than a lab.

This example illustrates one such embodiment.

### Decellularization

McIntosh apples were used for this experiment. The raw apples inspected, washed with tap water, and sanitized with a 0.1 ml/L chlorine solution. All of the kitchen instruments were thoroughly washed.

The washed apples were then peeled with a peeler, de-cored with a corer, cut into quarters, and chopped using a Hobart Buffalo chopper.

For the decellularization step all the chemicals utilized were food grade. 8 L of a 0.1% FCC Sodium Lauryl Sulphate (SLS) solution was prepared using a large Hobart stand mixer bowl equipped with a dough hook and operated at speed 1 using a dough hook. The solution was mixed for 24 hours.

The SLS solution and the chopped apples were transferred to a 10 L mixer bowl and were mixed for 24 hours using the Hobart stand mixer. After 24 hours, the mixer was stopped and the SLS was poured over a sieve. Another 8 L of 0.1% SDS solution was added to the mixing bowl and mixed for another 24 hours. This step was repeated once more.

After final mixing of the 0.1% SLS solution, the mixer bowl containing the chopped apples was filled with 8 L of tap water and subsequently poured out over the sieve in order to wash the apple. This step was repeated until no soapy residue remained (seven times).

After washing the mixing bowl with the tap water, the chopped apple and a 0.1M calcium chloride (FCC) solution prepared were added to the mixing bowl. The chopped apple and 0.1M calcium chloride solution were mixed with the dough hook at a speed of 1 as described above for 24 hours.

After mixing the chopped apples and the 0.1M calcium chloride solution, the mixture was poured over a sieve to retrieve the decellularized apple. The mixer bowl was filled with the decellularized apples and water and again poured out over the sieve. This step was repeated seven times in order to wash the calcium chloride residue from the decellularized apple.

### Mercerization

The decellularized material was manually pressed over a waste bucket to remove any water content therefrom. After pressing, the decellularized material was placed in a clean (dishwasher) pot.

For the mercerization step, all the chemicals utilized were food grade ones. A solution of 10% sodium bicarbonate (FCC) was prepared and added to the pot with the pressed decellularized material. The pot was placed on a gas stove with a kitchen exhaust hood on and heated to a temperature of about 80° C. A commercially available temperature probe was used to monitor the temperature of the heated solution.

To the heated solution was then added five aliquots of 25 mL of a 15% hydrogen peroxide stock solution. The solution was then manually stirred for 1 hour at 80° C.

After, the stove was turned off and the solution was placed in a fridge to cool. Using a pH probe, the solution was neutralized with a 50% citric acid solution until the pH was in the range of about 6.8 to about 7.2. Once neutralized, the solution was passed through a 25 μm stainless steel sieve. The sieved liquid was discarded. The sieved material was resuspended in water and neutralized and sieved again. This step was repeated until the pH of the sieved material was stabilized within the pH range of about 6.8 to about 7.2.

Once the pH of the sieved material stabilized, the material was sieved a final time for 1 hour and then passed through the sieve again to concentrate the material. The concentrated material was centrifuged at 8000 rpm for 15 minutes to produce the mercerized material. The supernatant was discarded and the mercerized material was vacuum sealed and stored in a fridge at 4° C.

### Scaffold Fabrication

The mercerized material was mixed with a 2% sodium alginate solution (texturing agent) at a ratio of 1:1. The material was then placed in silicone molds, which were then wrapped with plastic wrap and placed in a freezer overnight. The frozen material was then lyophilized in a Buchi L-200 lyophilizer at −55° C., 0.100 mbar for 48 hours.

A solution of 1% (w/v) calcium chloride dihydrate solution (crosslinking agent) (FCC) was prepared in accordance with the FCC. The lyophilized material was then crosslinked in a bath of 1% calcium chloride dihydrate solution overnight in a fridge at 4° C.

Thus, it is possible to produce a biomaterial scaffold for use in the methods of the present disclosure entirely in a kitchen using commercially available kitchen-grade equipment.

### Example 11: Characterization of Food-Grade Biomaterial Using Different Cooking Techniques

Six biomaterial scaffolds were manufactured in the same manner as Scaffold 2 of Example 1 without the C2C12 cells. Two scaffolds were cooked by pan-frying, two scaffolds were cooked via sous vide, and two scaffolds were baked.

For the pan-frying, scaffolds were pan-fried with vegetable oil over medium heat for 15 minutes. For the scaffolds cooked using sous vide, the scaffolds were cooked at 46° C. for 30 minutes and then seared to brown the surfaces. For the baking, the scaffolds were baked at 350° F. for 40 minutes.

The cooked samples are shown in FIG. 21, wherein FIG. 21A shows the sous vide samples, FIG. 21B shows the pan-fried samples, and FIG. 21C shows the baked samples.

### Results

The mass yields of the different cooking techniques are shown below in Table 4.

TABLE 4
Mass yields of biomaterial scaffolds
cooked using different techniques.

| Cooking Technique | Average Yield % | SD % |
|---|---|---|
| Sous vide | 74.29^{A} | 4.93 |
| Pan-cooking | 59.94^{B} | 4.45 |
| Baking | 32.87^{B} | 8.90 |

SD—standard deviation; Xlstat 2014
^{A-B}Means in a column without common superscripts are different (P < 0.05).

As shown, the different cooking techniques affected the mass yields of the cooked scaffolds.

In general, baking resulted in samples that were shrunk in size, not browned, and that had a dry exterior; pan-frying resulted in samples having a non-uniform shape and thus uneven browning; and sous vide resulted in samples that had little change in appearance after cooking (scaffold was translucent white) and browned during searing. All samples were gel-like internally after cooking.

### Example 12: Sensory Testing of Biomaterial Scaffolds Produced Using Kitchen-Safe Methods

3.5 kg of McIntosh Apples (43 apples) were decellularized and mercerized according to the procedure outlined in Example 10. In total, 854.5 g of mercerized apples was produced.

To produce the biomaterial scaffolds, 1 L of a 2% sodium alginate solution was prepared in the kitchen. The 854.5 g of mercerized apple was homogenized with 854.5 g of the 2% sodium alginate solution using a KitchenAid 4 qt Stand Mixer for 5 minutes at speed 6.

The homogenized scaffolds were then added to round, oval, and donut-shaped molds. The molds were put in a freezer for 24 hours and then lyophilized in a Buchi L-200 lyophilizer at −55° C., 0.100 mbar for 48 hours. Afterwards, the biomaterials were crosslinked with a calcium chloride solution for 1 hour.

### Sensory Testing

Sensory testing was completed in two stages. In the first stage, panelists were given two samples, one of which was a biomaterial scaffold sample, without knowing which sample was which. The panelists were then asked to determine which sample was the biomaterial scaffold.

Samples were cooked via deep-frying and sous vide (with subsequent searing in butter). For the deep-frying, the donut-shaped samples (chosen to mimic squid) were marinated in fish broth, dipped in a batter of wheat flour, rice flour, sodium bicarbonate, salt, pepper, and Perrier water, and then deep fried. For the sous vide samples, the round samples were cooked with fish broth at 46° C. for 30 min, after which the samples were seared in butter for about 2 minutes.

For the second stage of the sensory testing, the panelists were asked to describe the colour, odour, and tactile features of raw and cooked samples as compared to a reference. For the second stage, the cooked samples were prepared via sous vide as described above.

To judge the colour of the biomaterial samples, the panelists compared the raw and cooked samples to corresponding raw and cooked cod and squid samples. To judge the odour of the biomaterial samples, the biomaterial samples (raw and cooked “cod” and “squid”) were cut into small pieces and placed into cups with lids. Between samples, panelists cleansed their palate using coffee grounds.

To judge the tactile features of the samples, the panellists were given a knife and fork to compare the feel of the biomaterial samples (raw and cooked “cod” and “squid”) to actual cod and squid.

### Results

For the first stage of the sensory testing, nine panelists (4 Female and 5 Male, ranging from <20 to 50 years old) participated. From the panelists that participated on the adapted paired comparison test, over 50% of the total panelists were not able to get the perfect scoring combination for both cooking methods, with 28.54% of wrong answers in each cooking treatment.

For the second stage, descriptors were generated and the most frequently selected were used to describe the biomaterial scaffold samples as shown below in Table 5 and 6.

TABLE 5
Most frequent sensory parameter descriptors.
Sensory parameters descriptions

| raw colour | cooked colour | raw odour | cooked odour | raw tactile | cooked tactile |
|---|---|---|---|---|---|
| transparent/ | uniform colour (6) | metallic/ | metallic/ | wet/moist (6) | easy to |
| translucent (8) | transparent/ | astringent (4) | astringent (5) | hard to | cut/pierce (5) |
| porous/ | translucent (5) | no smell (4) | no smell (4) | cut/pierce (4) | squishy/spongy (4) |
| bubbles (5) | | sweet (2) | weaker than | squishy/spongy (4) | juicy/water |
| | | | uncooked (3) | juicy/water | release (3) |
| | | | | release (3) | skin layer (3) |
| | | | | easy to | wet/moist (3) |
| | | | | cut/pierce (2) | |

TABLE 6
Most frequent cooking technique descriptors.
Cooking methods comments

| Deep Fried | Sous Vide |
|---|---|
| Similar appearance (4) | Soft/mushy interior (8) |
| Less rubbery/easier cutting, tearing (4) | Not flaky (5) |
| Little/hard to see biomaterial (3) | Fries/potato wedges (4) |
| Perfect shape (2) | Similar smell (3) |
| Similar smell (2) | |

Thus, the unseeded biomaterial scaffolds are a suitable foundation for “lab-grown” meat products.

### Example 13: Taste-Testing of Biomaterial Scaffolds Produced Using Kitchen-Safe Methods

3.6 kg of McIntosh Apples (42 apples) were decellularized and mercerized according to the procedure outlined in Example 11 except without bleaching. In total, 767.5 g of mercerized apple was produced.

To produce the biomaterial scaffolds, 1 L of a 2% sodium alginate solution was prepared in the kitchen. 750 g of the mercerized apple was homogenized with 750 g of the 2% sodium alginate solution using a KitchenAid 4 qt Stand Mixer for 5 minutes at speed 6.

The homogenized scaffolds were then added to round and oval molds. The molds were put in a freezer for 24 hours and then lyophilized in a Buchi L-200 lyophilizer at −55° C., 0.100 mbar for 48 hours. Afterwards, the biomaterial scaffolds were crosslinked with a bath of a 1% (w/v) calcium chloride solution overnight in a fridge at 4° C.

### Taste-Testing

The biomaterial scaffolds were then cooked via sous vide at 50° C. for 30 minutes, baked at 400° F. for 25 minutes, or deep-fried at 375° F. (about 190° C.) for 2 minutes.

Seven panelists (4 Females and 3 Males, ranging from <20 to 50 years old) were tasked with describing the flavour and the texture of the cooked samples using a provided selection of descriptors. The descriptors were used to help in the characterization of the taste and texture of the samples.

### Results

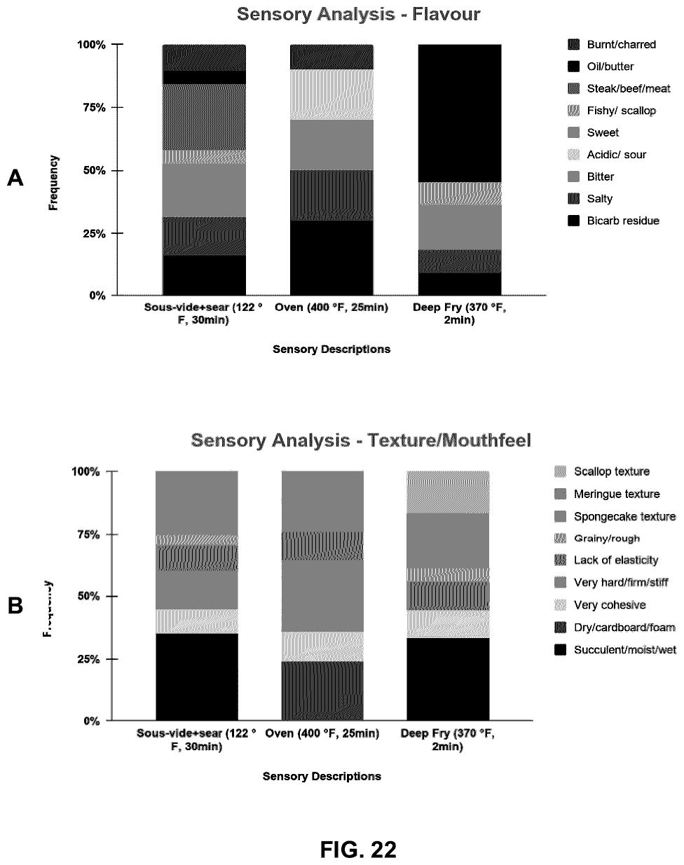

The results of the taste-testing are shown in FIG. 22, wherein FIG. 22A shows the distribution of descriptors used to describe the flavour of the samples, and FIG. 22B shows the distribution of descriptors used to describe the texture of the samples.

As shown, the beef flavour was the most frequent flavour observed in the sous vide-cooked scaffolds. Without being bound to a particular theory, it is thought that the beef flavour association may be due to the Maillard reaction that may occur after searing in butter (i.e. the reaction between the carbonyl group on a sugar and amino group). The oil/butter flavour was the most noticeable in the deep fry treatment, while a residual sodium bicarbonate taste, which may indicate that more washing steps could be used during sample preparation.

Further, in regards to texture, the characteristic most noted in the sous vide and deep-fried samples was succulence, while the characteristic most noted for the oven samples was dryness. In addition, a texture parameter detected in all three treatments was the cohesiveness.

Based on the results, the biomaterial scaffolds demonstrated the ability to efficiently absorb flavours while demonstrating an acceptable texture.

### Example 14: Colour Stability of Biomaterial Scaffolds

This study sought to investigate the stability of colouring agents added to the biomaterial scaffolds under various conditions. The colouring agents used in this study were beetroot powder (red) and sweet potato powder (purple), each provided by Suncore Foods.

### Colour Stability During Freeze-Drying

Mercerized apple samples were prepared according to the procedure outlined in Example 11. The mercerized apple samples were mixed with a sodium alginate solution to provide gels comprising 1% alginate. During mixing with the alginate solution, the samples were dyed to a final concentration of 3% beetroot powder, 3% of a beetroot and sweet potato powder mixture, or 3% sweet potato powder. The samples were then frozen overnight and subsequently lyophilized in a Buchi L-200 lyophilizer at −55° C., 0.100 mbar for 48 hours.

### Colour Stability in Water and Heat

Mercerized apple samples were prepared according to the procedure outlined in Example 11. The mercerized apple samples were mixed with a sodium alginate solution to provide gels comprising 1% alginate. During mixing with the alginate solution, the samples were dyed to a final concentration of 0.6% beetroot powder. After mixing, the sample was immediately crosslinked with a 1% calcium chloride solution.

After crosslinking, the sample was cooked via sous vide at 46° C. for 1 hour and any changes were noted. Biopsy punches of the samples were then placed in room temperature distilled water for 30 minutes or in 100° C. distilled water for 8 minutes. Biopsy punches were also taken from the samples prepared above for the freeze-drying analysis.

Colour Stability after Light Exposure

A sample was prepared as described above for the water and heat analysis.

After production, the sample was cut in half. One half was exposed to ambient light for four days, while the other half was kept in the dark for the four days.

### Colour Stability Using Different Concentrations of Mercerized Apple and Different Durations of Crosslinking

Three samples were prepared:
(1) 10% beetroot powder, 1% sodium alginate final concentration (62.5%), 37.5% mercerized apple, crosslinked for 15 minutes;
(2) 5% beetroot powder, 1% sodium alginate final concentration (50%), 50% mercerized apple, crosslinked for 15 minutes; and
(3) 5% beetroot powder, 1% sodium alginate final concentration (50%), 50% mercerized apple, crosslinked for 24 hours.

For each sample, the beetroot powder and the sodium alginate solution were first mixed and acidified to a pH of about 5.0. Then, the mixture was homogenized with the mercerized apple and crosslinked for the indicated time with a 1% calcium chloride solution.

Biopsy punches (1 cm) were taken from each sample after crosslinking and subjected to various temperatures. For sample (1), biopsy punches were taken and placed in boiling water (100° C.) for 8 minutes or in 50° C. water for 8 minutes. For sample (2), biopsy punches were added to 100° C., 80° C., or 60° C. water for 8 minutes. For sample (3), biopsy bunches were added to 100° C., 80° C., 60° C., or 40° C. water for 8 minutes, or maintained at room temperature for 20 minutes.

### Results

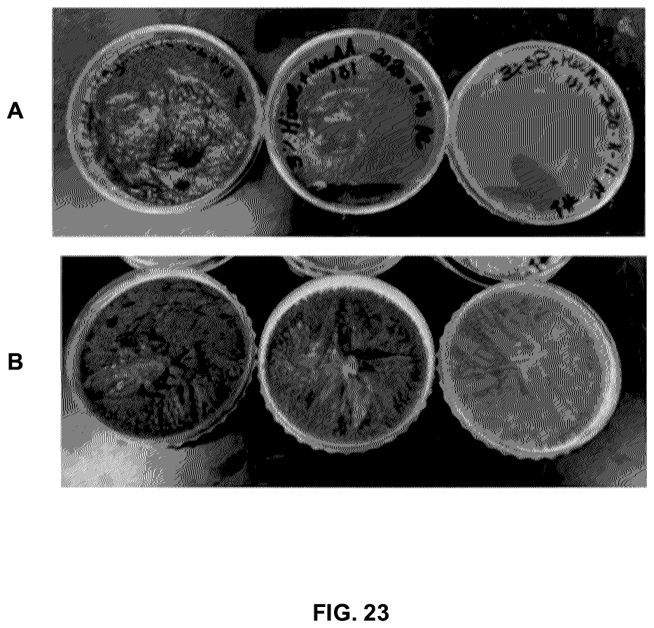

Photos of the samples of the “Colour Stability during Freeze-drying” study are shown in FIG. 23, wherein FIG. 23A shows the samples prior to freeze-drying and FIG. 23B shows the samples after freeze-drying.

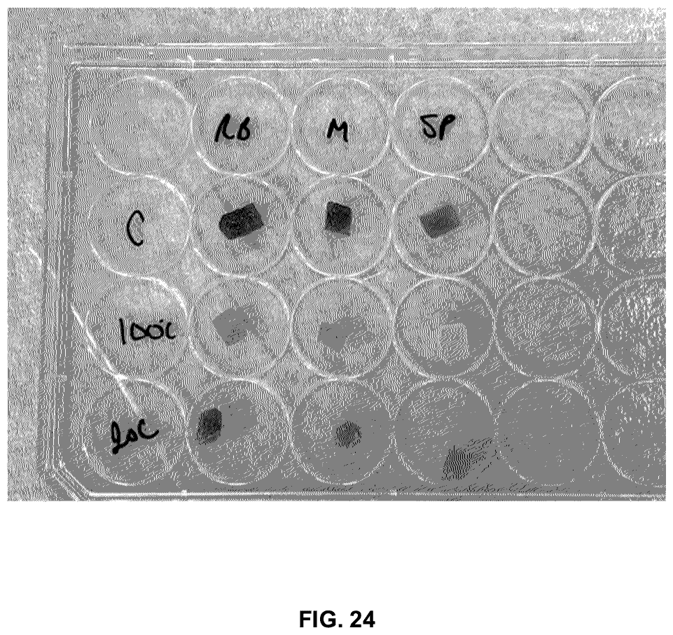

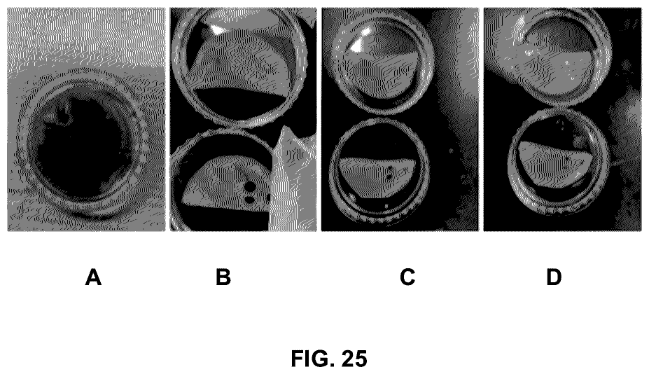

Photos of the samples after the “Colour Stability in Water and Heat” are shown in FIG. 24, wherein RB refers to the samples having only the red beetroot powder, M refers to the samples having a mix of the beetroot and sweet potato powder, SP refers to the samples having only the sweet potato powder, and C refers to the samples not exposed to water.

Photos of the samples of the “Colour Stability after Light Exposure” study are shown in FIG. 25, wherein FIG. 25A shows the two halves of the sample before treatment, FIG. 25B shows the two halves after day 2, FIG. 25C shows the two halves after day 3, FIG. 25D shows the two halves after day 4. It is noted that in each photo, the bottom half of the sample was the half that was not exposed to light.

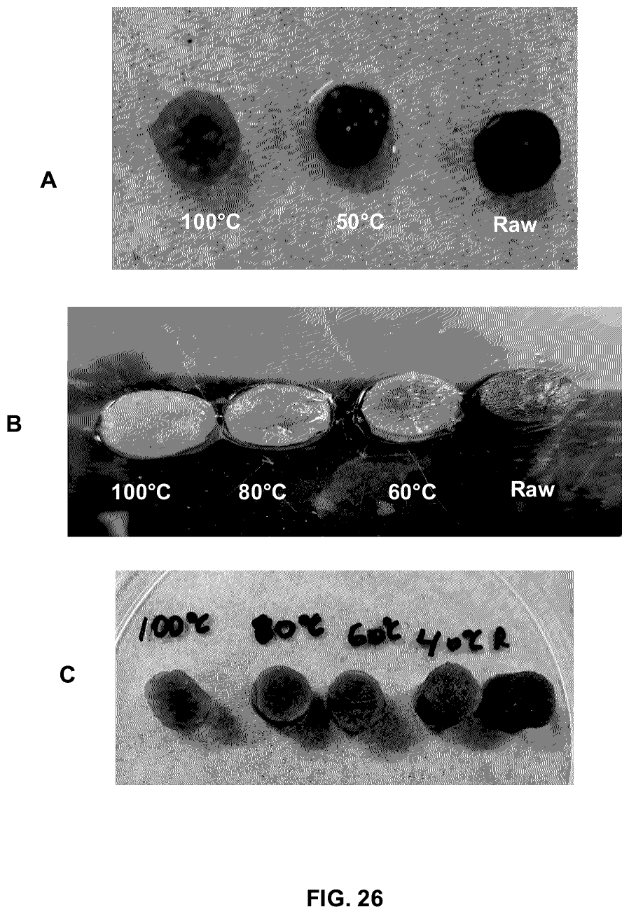

Photos of the samples after the “Colour Stability using different concentrations of Mercerized Apple and different durations of Crosslinking” study are shown in FIG. 26, wherein FIG. 26A shows the biopsy punches of sample (1), FIG. 26B shows the biopsy punches of sample (2), and FIG. 26C shows the biopsy punches of sample (3).

As shown, generally, the colour of the samples degraded at higher temperatures and over time whether or not the samples were exposed to light. However, it was also shown that a mixture that was 50% alginate solution and beetroot powder and 50% mercerized apple that is subsequently crosslinked for 24 hours may maintain the colour of the material.

### Example 15: Use of Fibrous Mercerized Tissue to Produce Biomaterial Scaffolds

This study sought to investigate the use of mercerized fibrous tissue to mimic the fibers found in meat products. Palm hearts were selected to provide mercerizable fibrous tissue.

The palm hearts were cut either longitudinally or transversely. After cutting, the palm hearts were decellularized by being placed in separate beakers containing a 0.1% SDS solution. The palm hearts were immersed for three days and the SDS solution was changed daily. After, the palm hearts were washed three times with distilled water. The palm hearts were then immersed in a 0.1M calcium chloride solution for 24 hours, after which the palm hearts were washed with distilled water.

A portion of the palm hearts were then mercerized using the procedure outlined above in Example 9 using a 10% sodium bicarbonate solution, a 15% hydrogen peroxide stock solution, and acetic acid.

70 g of the mercerized longitudinally cut palm hearts were then mixed with 50 g of the decellularized longitudinally cut palm hearts and 70 g of a 2% sodium alginate solution using a whisk. The mixture was then transferred to silicone molds, frozen for 48 hours, and lyophilized for 40 hours at 0.100 mbar and −55° C.

The lyophilized mixture was then crosslinked using a 1% calcium chloride solution in fish broth for 30 minutes.

The crosslinked mixture was then pan-fried. Square-shaped mixtures were used to mimic fish sticks, while round-shaped mixtures were used to mimic scallops.

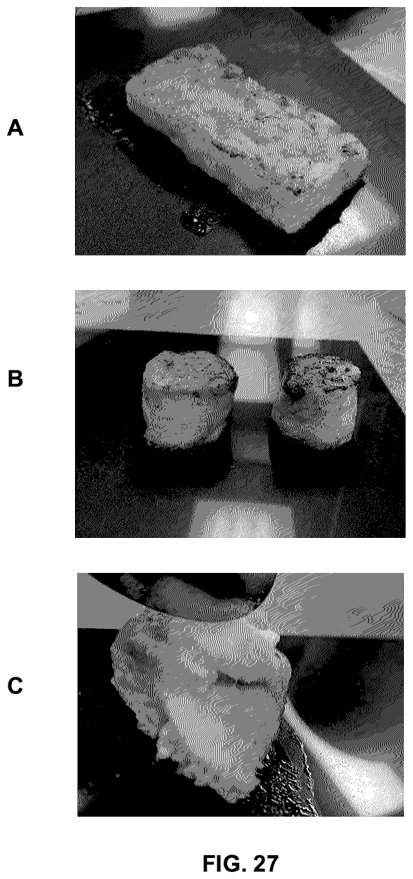

The resulting foods are shown in FIG. 27, wherein FIG. 27A shows an imitation fish stick, FIG. 27B shows an imitation scallop, and FIG. 27C shows a layer of the imitation fish stick peeled back. As shown, the imitation food products resemble their natural meat counterparts.

### Example 16: Unidirectional Freezing of Biomaterial Scaffolds

This study sought to investigate whether unidirectional freezing of biomaterial scaffolds may produce biomaterial scaffolds having an aligned, porous structure resembling meat fibres.

To investigate the effects of unidirectional freezing, three samples were prepared as outlined below.

### Sample 1

A biomaterial scaffold was prepared using 7.5 g of mercerized apple and 7.5 g of a 2% sodium alginate solution. The biomaterial scaffold was positioned in a styrofoam support and then placed in a unidirectional freezer for 3 hours. After the unidirectional freezing, the treatment was transferred to a conventional freezer for 48 hours. The sample was then lyophilized at 0.100 mbar and −55° C. for a further 48 hours.

The sample was then imaged under a microscope.

### Sample 2

The biomaterial scaffold was prepared using a 1:1 ratio of mercerized apple and 2% sodium alginate solution. The biomaterial scaffold (20 mL) was added to a petri dish.

The sample had a height of about 1 mm. The petri dish was placed in the unidirectional freezer for 3 hours. After the unidirectional freezing, the treatment was transferred to a conventional freezer for 48 hours. The sample was then lyophilized at 0.100 mbar and −55° C. for a further 48 hours.

The sample was then imaged under a microscope.

### Sample 3

Two bio material scaffolds were prepared using a 1:1 ratio of mercerized apple and 2% sodium alginate solution. One of the samples (sample 3B) had 20 g of red beetroot powder added to 20 mL of the 2% sodium alginate solution during production thereof. 30 mL of each of the samples were then added to inox mold containers. The containers were placed in the unidirectional freezer for 4 hours. After the unidirectional freezing, the treatment was transferred to a conventional freezer for 48 hours. The sample was then lyophilized at 0.100 mbar and −55° C. for a further 48 hours.

The sample was then imaged under a microscope.

### Results

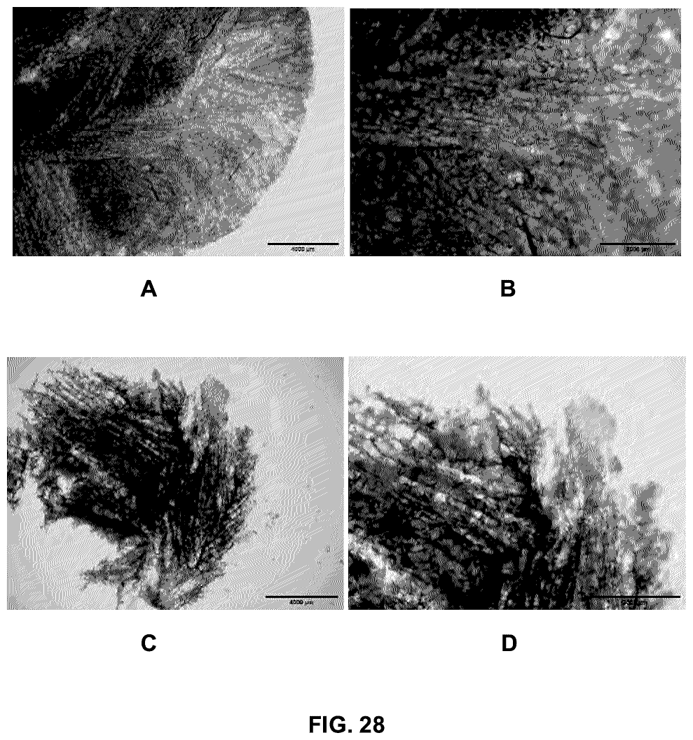

FIG. 28 shows microscopy images of Sample 1 of this Example, wherein FIG. 28A shows a microscopy image of the top side of the sample at 0.7×, FIG. 28B shows a microscopy image of the top side of the sample at 1.6×, FIG. 28C shows a microscopy image of the bottom side of the sample at 0.7×, and FIG. 28D shows a microscopy image of the bottom side of the sample at 1.6×.

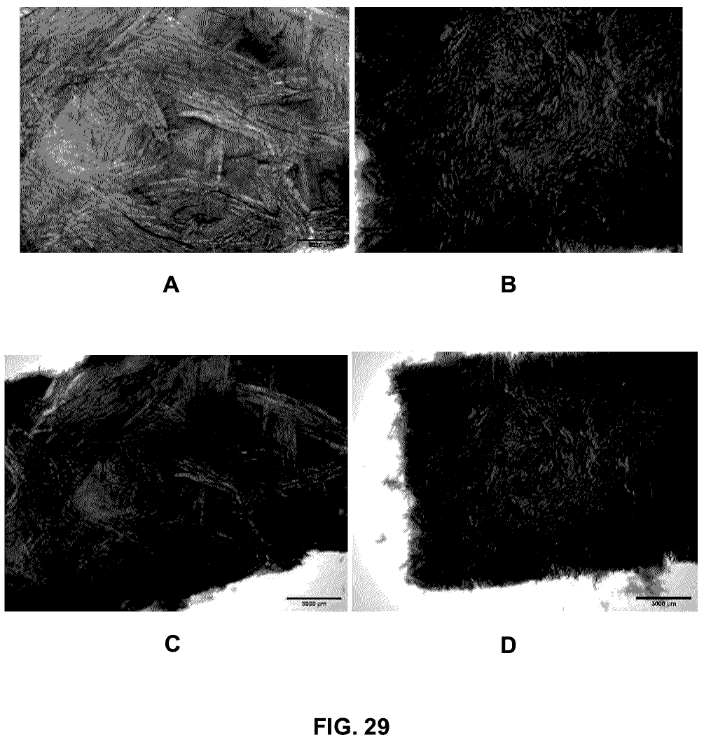

FIG. 29 shows microscopy images of Sample 2 of this Example, wherein FIG. 29A shows a microscopy image of an edge of the sample at 0.7×, FIG. 29B shows a microscopy image of the centre of the sample at 0.7×, FIG. 29C shows a microscopy image of the edge of the sample at 1.0×, and FIG. 29D shows a microscopy image of the centre of the sample at 1.0×.

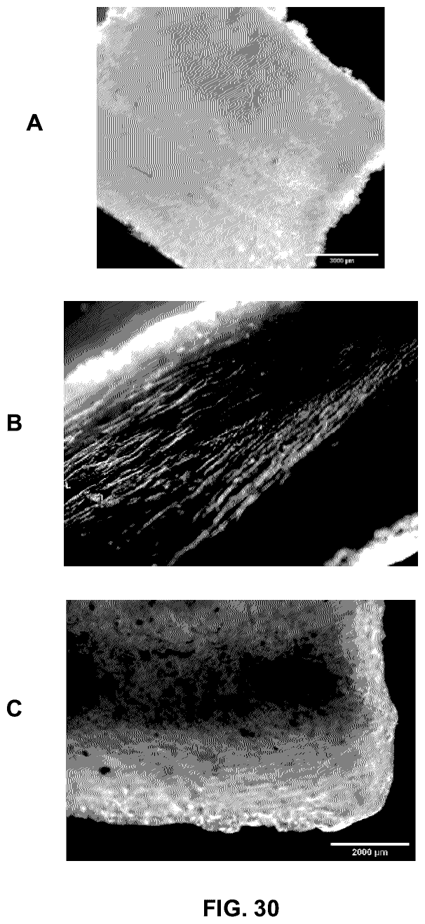

FIG. 30 shows microscopy images of Sample 3 of this Example, wherein FIG. 30A shows a microscopy image of a top surface of the sample 3A (not dyed with beetroot powder) at 1.0×, FIG. 30B shows a microscopy image of a side of the sample 3B after lyophilization at 1.6×, and FIG. 30C shows a microscopy image of a corner of the sample 3B at 1.6×.

As shown, each of the samples produced had a generally aligned porous structure. However, the sample 3B, as shown in FIG. 30B, had an aligned porous structure that closely resembled that of a natural meat product. Without being bound to a particular theory, it is thought that the inox mold material may affect the formation of such aligned porous structures. As well, it is noted that the addition of the dye (the beetroot powder) may make the porous-like fibers more evident.

### Example 17: Use of Different Adhering Agents to Mimic Muscle

This study sought to investigate the use of adhering agents to adhere biomaterial scaffold portions together in order to mimic the structure of muscle.

For this study, two samples were prepared. A first sample was prepared in the same manner as sample 3B of Example 17. A second sample was prepared using a 1:1 ratio of mercerized apple and 2% sodium alginate solution.

The first sample was cut into four separate portions. Two of the portions were adhered together using a thin layer of a 2% sodium alginate solution as an adhering agent to produce two imitation meat products. One of the portions was crosslinked using a 1% calcium chloride solution for 1 hour at room temperature, while the other was crosslinked for 24 hours in a fridge. The portion that was crosslinked for one hour was then pan-fried in butter for 1 minute on each side, while the portion crosslinked for 24 hours was cooked in 100° C. water for 8 minutes.

The second sample was divided into five separate replicates during production to produce five separate layers of biomaterial scaffold. The layers were then each placed in a 60 mm petri dish and frozen for 48 hours. The layers were then lyophilized at 0.100 mbar and −55° C. and adhered using a thin layer of a 2% sodium alginate solution. The sample was then crosslinked using a 1% calcium chloride solution for 1 hour at room temperature. After crosslinking, the sample was pan-fried in butter for 1 minute on each side.

### Results

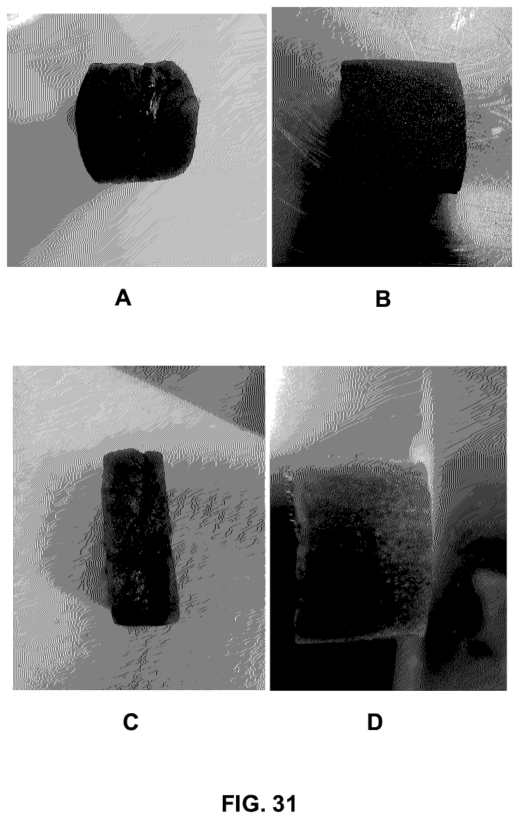

The portions of the first sample are shown in FIG. 31, wherein FIG. 31A shows a photograph of a side of the portion crosslinked for 1 hour, FIG. 31B shows a photograph of a side of the portion crosslinked for 1 hour after cooking, FIG. 31C shows a photograph of a side of the portion crosslinked for 24 hours, and FIG. 31D shows a photograph of a side view of the portion crosslinked for 24 hour after cooking.

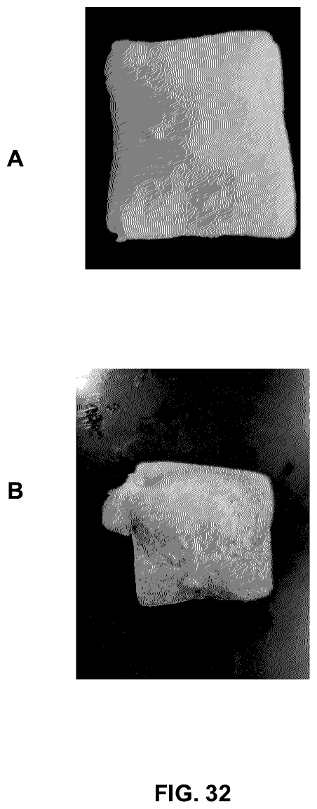

The second sample is shown in FIG. 32, wherein FIG. 32A shows a photograph of a top side of the second sample after crosslinking, and FIG. 32B shows a photograph of the top side of the second sample after pan frying.

As shown, the 2% sodium alginate solution demonstrated efficacy to glue together pieces and/or layers of lyophilized biomaterial. In addition, the sodium alginate as a glue was able to maintain the scaffold during pan-frying and boiling.

### Example 18: Use of Transglutaminase (TGM) as a Crosslinker

This study sought to explore the efficacy of TGM as a crosslinker in the methods of the present disclosure. In this study, a biomaterial scaffold was constructed using pea protein.

Two trials were completed and are outlined below.

### Trial 1

A biomaterial scaffold comprising 20% (w/w) isolated pea protein (having 90% protein content), 75% (w/w) mercerized apple, and 5% (w/w) TGM was prepared. The isolated pea protein and the mercerized apple were first mixed either by (1) whisking or (2) blending. Then, the mixed isolated pea protein and the mercerized apple were cooked via sous vide for 20 minutes at 40° C. The TGM was then added to the isolated pea protein and mercerized apple and the mixture was cooked via sous vide for a further 4 hours at 40° C. Afterwards, the samples were placed in a fridge at 4° C. for three days.

### Trial 2

A first biomaterial scaffold comprising 20% (w/w) isolated pea protein (having 90% protein content), 74% (w/w) mercerized apple, 1% (w/w) water, and 5% (w/w) TGM, and a second biomaterial scaffold comprising 20% (w/w) isolated pea protein (having 90% protein content), 74% (w/w) mercerized apple, 5% (w/w) water, and 1% (w/w) TGM were prepared. Both scaffolds were prepared by mixing with a whisk.

The scaffolds were cooked via sous vide for 1 hour at 40° C. Afterwards, the scaffolds were subjected to a heat stability test wherein the scaffolds were placed directly into boiling water for 8 minutes.

### Results

The products obtained in Trial 1 demonstrated the following sensory characteristics: buttery smell, granular mouthfeel (breaks into small, chewy chunks), chewy, no noticeable bitterness, and slightly harder to cut with a fork after cooking. For Trial 2, both concentrations of TGM crosslinked the samples. As well, the products obtained in Trial 2 demonstrated the following characteristics: pea protein flavour, firm gel texture, soft sausage texture, uniform particles (more in the 1% treatment), and no bitter flavour.

### Example 19: Recipes Incorporating Biomaterial Scaffolds

This study sought to investigate various recipes incorporating bio material scaffolds of the present disclosure therein.

### Pulled Pork Sandwich

Palm hearts were torn into longitudinal strands and decellularized using the procedure outlined in Example 16.

A barbeque sauce was produced using 475 mL tomato sauce, 120 mL apple cider vinegar, 120 mL honey, 60 mL molasses, 45 mL Worcestershire sauce, 15 g smoked paprika, 15 g garlic powder, 7.5 g black pepper, 7.5 g onion powder, and 7.5 g fine sea salt.

The decellularized palm heart and the barbeque sauce were spread in separate trays and cold smoked for 15 minutes. After, decellularized palm heart was added to the barbeque sauce and cooked via sous vide for 1 hour at 40° C.

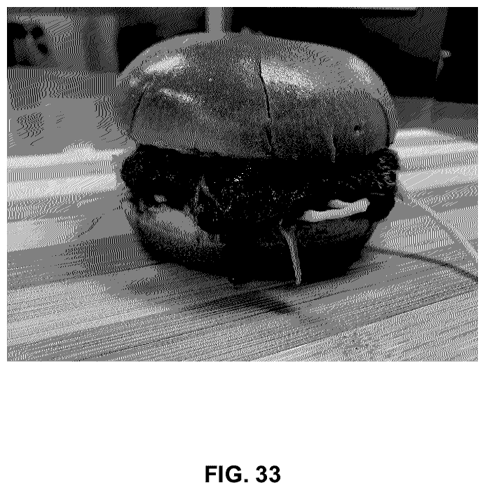

The resulting imitation pulled pork was added to a bun with coleslaw. The finished sandwich is shown in FIG. 33. In general, the imitation pulled pork was softer and more watery than actual pulled pork. As well, the palm heart absorbed a significant amount of smoke and barbeque sauce flavour, which masked the flavour of the palm heart.

### Foie Gras

A number of biomaterial scaffold formulations were prepared to investigate which formulation best mimicked foie gras. The formulations produced are shown below in Table 7.

TABLE 7
Foie gras formulations.

| Ingredients | F1 | F2 | F3 | F4 | F5 |
|---|---|---|---|---|---|
| Mercerized apple (g) | 34 | 48 | 94 | 63 | 78 |
| Pea protein (g) | 10 | 19 | 20 | 15 | 30 |
| Sunflower oil (mL) | 5 | 12.72 | 20 | 20 | 25 |
| Coconut oil (mL) | | | 50 | 20 | 25 |
| TGM (g) | 0.5 | 1 | 2 | 1 | 2 |
| NaCl (g) | 0.5 | 0.09 | 2 | 1 | 2 |
| Coconut milk (mL) | — | 36.29 | — | — | — |
| Sodium Alginate - 2% | — | — | — | 4 | 3 |
| (mL) | | | | | |
| Iota- Carrageenan (mL) | — | — | 2 | — | — |
| Carrageenan + LB (mL) | — | 1 | — | — | — |
| Caramel colour (g) | — | — | 0.5 | — | — |
| Red oxidase colour (g) | — | — | 0.5 | — | — |
| Turmeric (g) | — | — | — | 0.5 | 0.1 |
| Sous Vide | 86° C./ | 50° C./ | 86° C./ | 50° C./ | 50° C./ |
| | 1 h | 1 h | 2 h | 3 h | 2 h |
| | | 60° C./ | | | |
| | | 1 h | | | |

For each formulation, the mercerized apple was mercerized using a 10% sodium bicarbonate solution, except for the mercerized apple of formulation F2, which was mercerized using 1M NaOH. As well, during preparation of formulation F2, the coconut milk and mercerized apple were first mixed to form a mousse-like mixture prior to the addition of the remaining ingredients.

After preparing each of the formulations, the formulations were then cooked via sous vide using the parameters shown in Table 7 above. After cooking via sous vide, the formulations were pan-fried to brown the surfaces thereof.

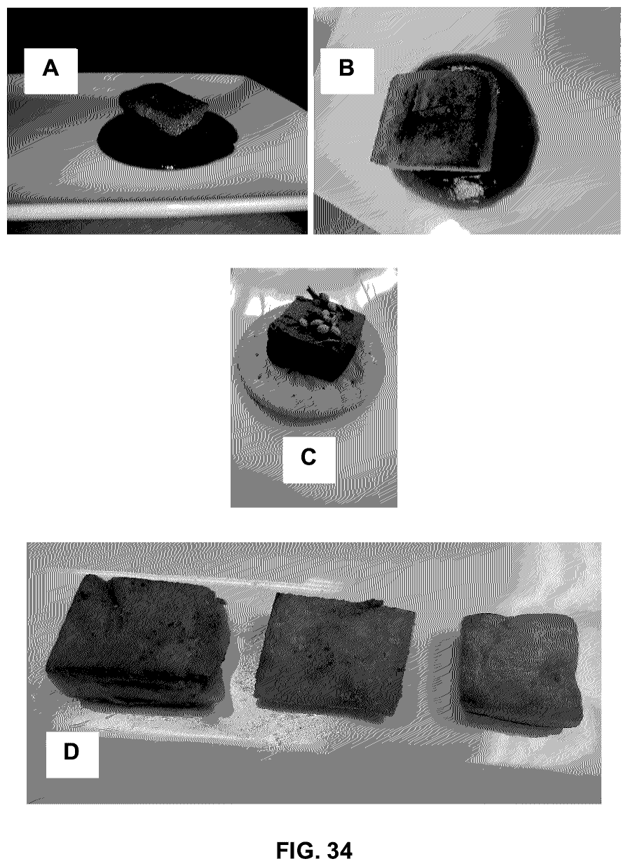

FIG. 34 shows the resulting imitation foie gras, wherein FIG. 34A shows a photograph of the plated formulation F1, FIG. 34B shows a photograph of the plated formulation F2, FIG. 34C shows a photograph of the formulation F3 on a cracker, and FIG. 34D shows a photograph of the formulations F4 (left) and F5 (middle) compared to actual foie gras (right). As shown, the biomaterial scaffold formulations closely resemble actual foie gras.

Further, the mechanical properties of the actual foie gras were compared to those of formulations F1 and F2. 1 cm biopsy punches were taken from raw and cooked foie gras and formulations F1 and F2. The mechanical properties of the punches were analyzed using a UniVert Mechanical Test System having a 1N load cell installed therein. The settings for the test were 90% compression and a compression rate of 5%/s.

The stiffness of the raw and cooked samples are shown below in Tables 8 and 9, respectively.

TABLE 8
Stiffness of raw formulations F1 and F2 and foie gras.

Stiffness (Kpa)

| Treatment | Average | SD |
|---|---|---|
| Raw Foie Gras | 4.54^{A} | 1.92 |
| Raw FA | 7.17^{A} | 2.74 |
| Raw FE | 10.59^{A} | 6.37 |

SD—standard deviation; Xlstat 2014
No statistical difference (P > 0.05) was observed among the treatments (Raw FG, FA, and FE).

TABLE 9
Stiffness of cooked formulations F1 and F2 and foie gras.

Stiffness (Kpa)

| Treatment | Average | SD |
|---|---|---|
| Cooked Foie Gras | 20.32^{A} | 0.48 |
| Cooked FA | 23.91^{A} | 0.88 |
| Cooked FE | 6.30^{B} | 3.46 |

SD—standard deviation; Xlstat 2014.
^{A-B}Means in a column without common superscripts are different (P < 0.05).

As shown, the formulation FA, both raw and cooked, presented similar stiffness compared to actual foie gras. Without being bound to any particular theory, it is thought that the similarities in stiffness may be due to the protein content about (20%) of the formulation FA.

### Fish Fillet

Palm hearts were torn into longitudinal strands and decellularized using the procedure outlined in Example 16, except over a period of 5 days instead of 3 days. The decellularized palm hearts were used for two different formulations.

Both formulations comprised the decellularized palm heart, 78 g of mercerized apple, 15 g of pea protein, 5 mL of sunflower oil, 9 mL of sodium alginate, 1 g of NaCl, 1 g of transglutaminase, and 0.1 g of turmeric.

After preparation, a first formulation was then cooked via sous vide for 3 hours at 50° C., while a second formulation was placed in an inox mold, vacuum-sealed, frozen for 24 hours, lyophilized for 48 hours, crosslinked with 1% calcium chloride, and then cooked via sous vide for 3 hours at 50° C.

After the cooking via sous vide, both formulations were pan-fried for one minute on each side.

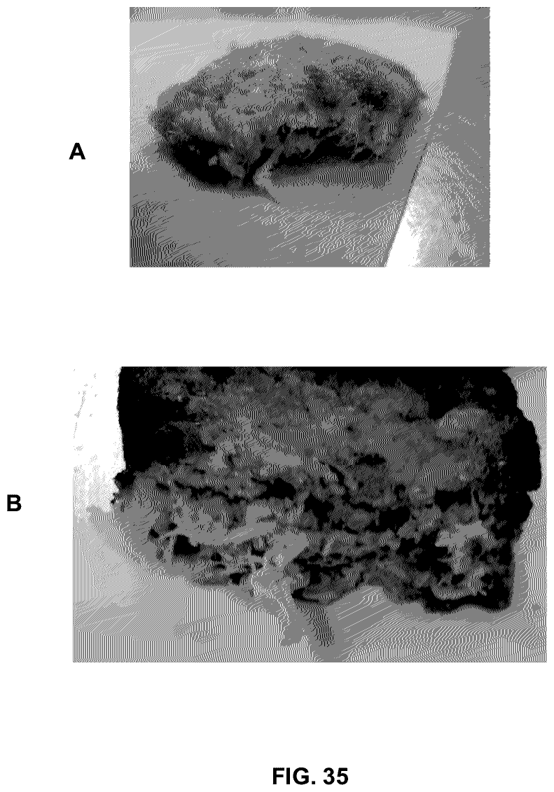

The resulting fillets are shown in FIG. 35, wherein FIG. 35A shows a photograph of the first formulation cut open and FIG. 35B shows a photograph of the second formulation cut open.

The second imitation fish fillet formulation comprised the decellularized palm heart, 78 g of mercerized apple, 15 g of pea protein, 5 mL of sunflower oil, 9 mL of sodium alginate, 1 g of NaCl, 1 g of transglutaminase, and 0.1 g of turmeric. After preparation, the formulation was then cooked via sous vide for 3 hours at 50° C.

As shown, both formulations produced an imitation fish fillet having a fibrous structure.

### Example 20: Histological Analysis of Seeded Biomaterial Scaffolds of the Present Disclosure and Various Types of Meat Products

C2C12 myoblast cells were grown in 30 cell plates until a confluency of 100% was achieved. A cell pellet formed thereby was then re-suspended in 200 μL of DMEM (10% FBS and 1% Penicillin/Streptomycin).

3 mL of a mixture containing 1.5 mL of a 2% sodium alginate solution (final concentration of 1%), 1.5 mL of mercerized apple (centrifuged), and 200 μL of 1.4×10⁸ C2C12 myoblast cells was prepared via mixing in a syringe as outlined in Example 2.

The mixture was homogenized 20 times between two 3 mL syringes and then divided into 1 mL replicates in three separate wells of a 24-well plate. In total, there were about 4.6×10⁷ cells for each well.

The mixtures were then crosslinked using a 1% CaCl₂) dihydrate solution for 24 hours in a fridge. After the crosslinking, the mixtures were vacuum-sealed and maintained in the fridge for a further 24 hours.

After the mixtures were maintained under vacuum for 24 hours, the resulting seeded biomaterial scaffolds were washed three times with PBS, fixed with 4% PFA in PBS for 1 hour, washed an additional three times with PBS, and then kept in 70% EtOH until the histological analysis.

Three different types of meat products were also prepared for the histological analysis. Beef samples, tuna samples, and scallop samples were each cut longitudinally or perpendicularly to the fibers thereof. Each sample was then washed three times with PBS, fixed with 4% PFA in PBS for 72 hours, washed an additional three times with PBS, and then kept in 70% EtOH until the histological analysis.

The seeded biomaterial scaffolds and the meat samples were stained with hematoxylin and eosin (HE) or Masson's Trichrome (MT) and then analyzed under microscope.

### Results

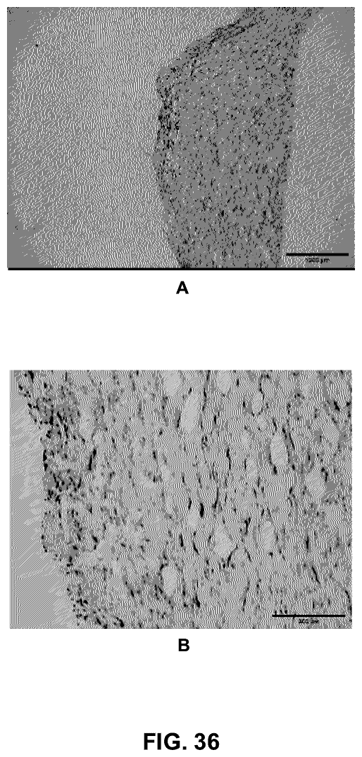

The results are shown in FIG. 36 to FIG. 45.

FIG. 36 shows microscopy images of a seeded biomaterial scaffold stained with HE, wherein FIG. 36A shows the HE-stained seeded biomaterial scaffold at a magnification level of 2.5×, and FIG. 36B shows the HE-stained seeded biomaterial scaffold at a magnification level of 10×.

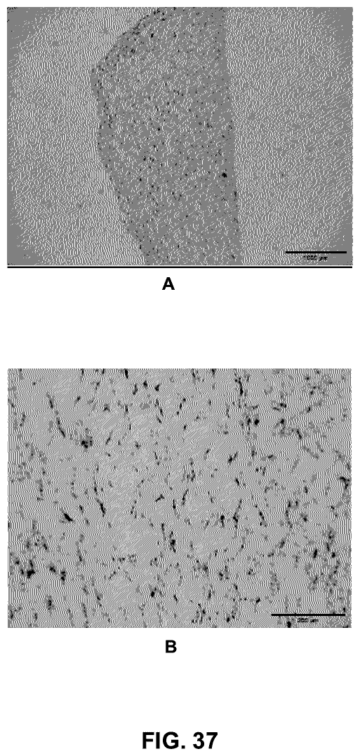

FIG. 37 shows microscopy images of a seeded biomaterial scaffold stained with MT, wherein FIG. 37A shows the MT-stained seeded biomaterial scaffold at a magnification level of 2.5×, and FIG. 37B shows the MT-stained seeded biomaterial scaffold at a magnification level of 10×.

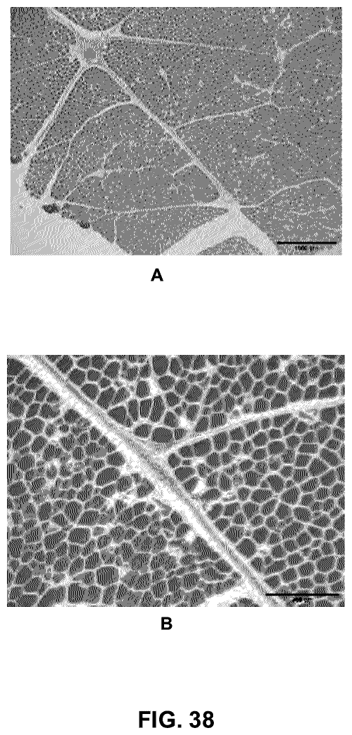

FIG. 38 shows microscopy images of a beef sample cut perpendicularly relative to the fibers thereof stained with HE, wherein FIG. 38A shows the HE-stained beef sample at a magnification level of 2.5×, and FIG. 38B shows the HE-stained beef sample at a magnification level of 10×.

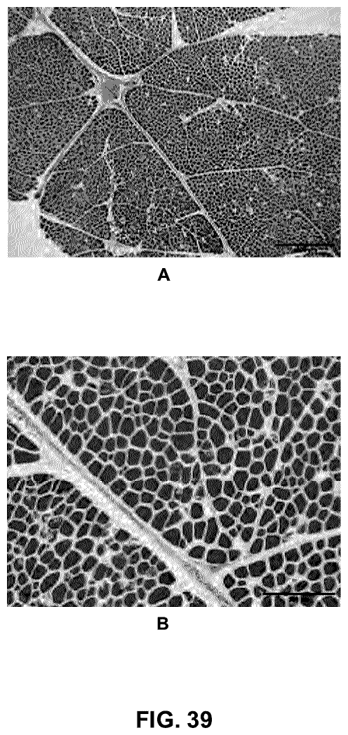

FIG. 39 shows microscopy images of a beef sample cut perpendicularly relative to the fibers thereof stained with MT, wherein FIG. 39A shows the MT-stained beef sample at a magnification level of 2.5×, and FIG. 39B shows the MT-stained beef sample at a magnification level of 10×.

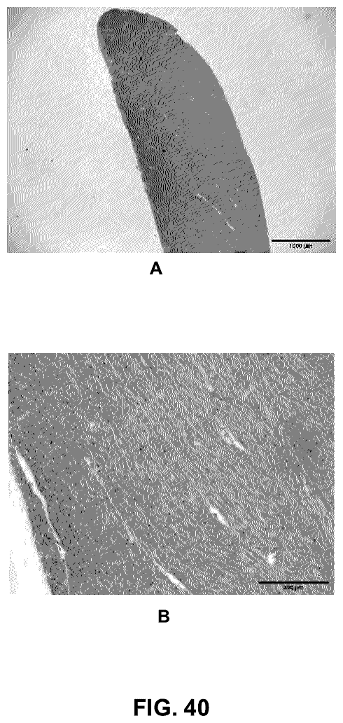

FIG. 40 shows microscopy images of a scallop sample cut longitudinally relative to the fibers thereof stained with HE, wherein FIG. 40A shows the HE-stained scallop sample at a magnification level of 2.5×, and FIG. 40B shows the HE-stained scallop sample at a magnification level of 10×.

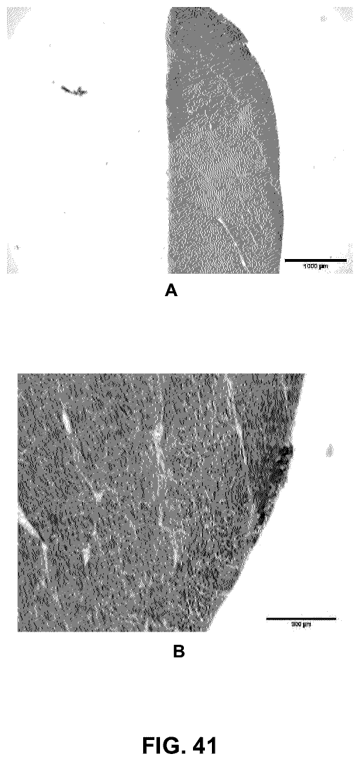

FIG. 41 shows microscopy images of a scallop sample cut longitudinally relative to the fibers thereof stained with MT, wherein FIG. 41A shows the MT-stained scallop sample at a magnification level of 2.5×, and FIG. 41B shows the MT-stained scallop sample at a magnification level of 10×.

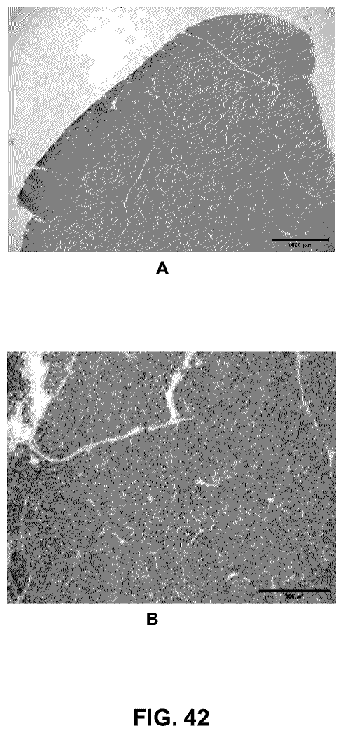

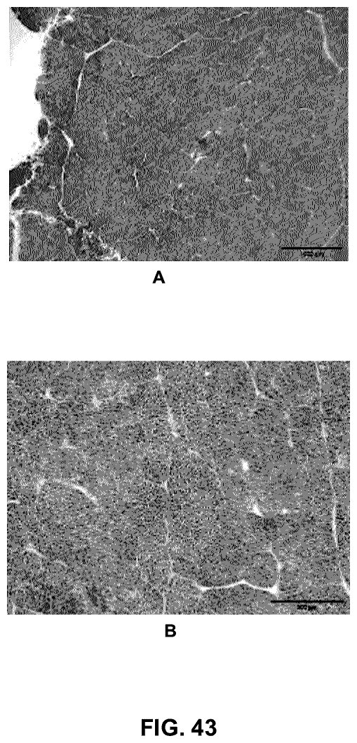

FIG. 42 shows microscopy images of a scallop sample cut perpendicularly relative to the fibers thereof stained with HE, wherein FIG. 43A shows the HE-stained scallop sample at a magnification level of 2.5×, and FIG. 43B shows the HE-stained scallop sample at a magnification level of 10×.

FIG. 43 shows microscopy images of a scallop sample cut perpendicularly relative to the fibers thereof stained with MT, wherein FIG. 43A shows the MT-stained scallop sample at a magnification level of 2.5×, and FIG. 43B shows the MT-stained scallop sample at a magnification level of 10×.

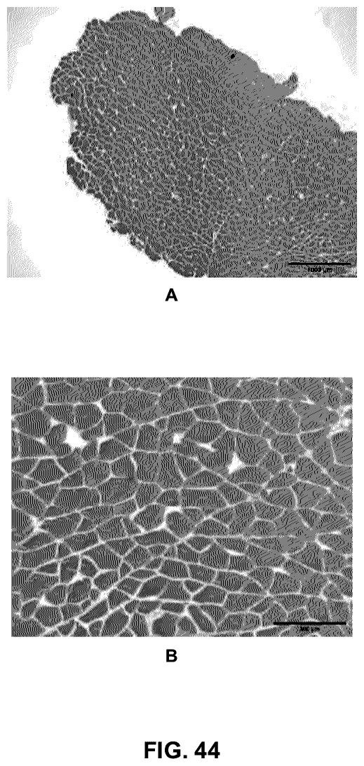

FIG. 44 shows microscopy images of a tuna sample cut perpendicularly relative to the fibers thereof stained with HE, wherein FIG. 44A shows the HE-stained tuna sample at a magnification level of 2.5×, and FIG. 44B shows the HE-stained tuna sample at a magnification level of 10×.

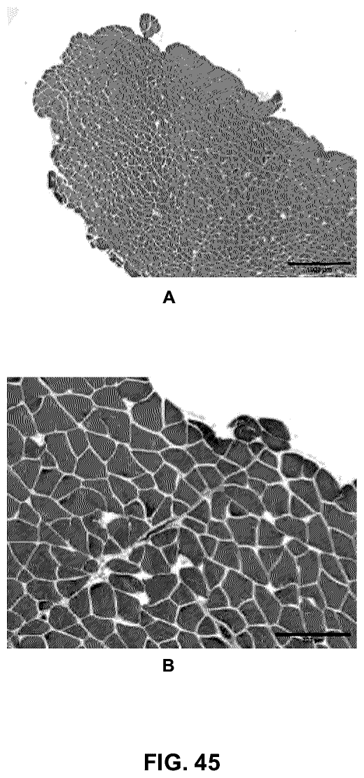

FIG. 45 shows microscopy images of a tuna sample cut perpendicularly relative to the fibers thereof stained with MT, wherein FIG. 45A shows the MT-stained tuna sample at a magnification level of 2.5×, and FIG. 45B shows the MT-stained tuna sample at a magnification level of 10×.

As shown, while there are differences, the seeded biomaterial scaffolds produced by the methods of the present disclosure have largely uniform cellular structures similar to those of the various meat samples.

### Example 21: Vegan Sashimi Comprising Seeded Biomaterial Scaffolds Produced by the Methods of the Present Disclosure

This Example describes an example of a vegan sashimi recipe incorporating seeded biomaterial scaffolds produced by the methods of the present disclosure.

The vegan sashimi was formulated using the following ingredients:
50 g of mercerized apple
50 mL of water
3 g of Konjac flour
1 g of sodium alginate
1 g of sodium chloride
2.5 g of red beet powder
2.5 g of purple sweet potato powder
0.6 g of titanium dioxide
0.1 g of red iron oxidase

The above ingredients were mixed without the mercerized apple in a stand mixer and subsequently homogenized (to reduce lumps therein) using a hand blender. The mercerized apple was then added to the homogenized mixture and the mixture was re-homogenized using a stand mixer with a whisk pedal.

The mixture was then placed in a rectangular silicone mold and crosslinked on each side for 30 minutes using a 0.75% CaCl₂) solution. The crosslinking produced a soft gel that was then transferred to a silicone container and further crosslinked in a fridge for 24 hours.

After 24 hours, the vegan sashimi was wrapped in seaweed (nori) and cooked via sous vide at 50° C. for 1 hour.

### Results

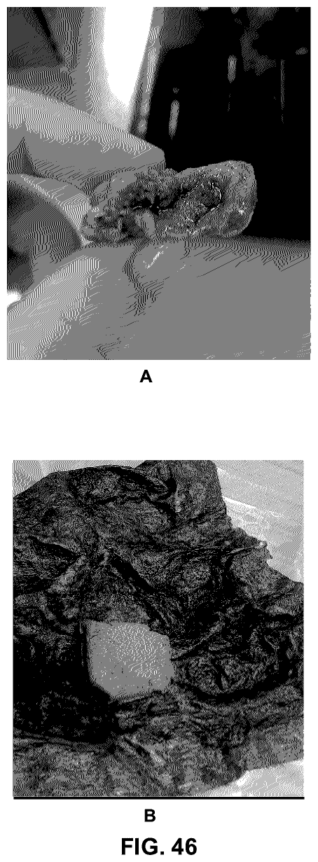

FIG. 46A shows a photograph of a cross-section of the vegan sashimi before cooking via sous vide, and FIG. 46B shows a photograph of the cooked vegan sashimi. As shown, the finished product closely resembles actual sashimi.

### Example 22: Plant Based Salmon Prototype—Experiment 1: Nutritional Composition of the Plant-Based Salmon Prototype Infiltrated with Chinook Salmon Cells (CHSE-214) Using Combined Techniques—Vacuum and Mixing

The objective of this experiment was to characterize the nutritional composition of the plant-based salmon fillet prototype infiltrated with Chinook salmon cells, instead of C2C12 cells, by conducting two trials.

### Trial 1

The plant-based salmon fillet was formulated using the following ingredients:
73 g water
35.5 g mung bean isolate
20 g floss mung bean TVP
16.5 g wheat TVP
26.5 g sunflower oil
2 g konjac gum
3 g tapioca starch
6 g 0.5M Ca(OH)₂
1 g salt
0.15 g CaSO₄
105 g merAA
1.2 g salmon flavour
0.9 g flavour masker
0.04 g ExBerry brilliant orange (liquid)
0.04 g Astaxanthin
0.15 g Watermelon pink (liquid)
0.6 g Glucone delta lactone

The formulation was treated with Chinook Salmon cells and PBS and the following concentrations were used:
Formulation—97.1 g (92.4%)
Chinook Salmon (CHSE-214 Cells)—5.59 g (5.55%)
PBS—2.4 g (2.05%)
Total—105.09 g (100%)

The formulation was analysed for Humidity, Ash, Protein, Lipid, Sodium, Cholesterol, and Total sugars content.

### Protocol

All the ingredients were weighed. Out of all the ingredients, mung bean and wheat TVP were soaked in hot water for 30 minutes in separate bowls. Water was then squeezed out from the soaked TVP. All ingredients were mixed, except Ca(OH)₂ and the 2 TVPs, in a thermomixer to form a paste. The paste was then heated in the microwave for 20 seconds, followed by which, 6 g 0.5M Ca(OH)₂ were then added and mixed into the paste in a thermomixer. The shredded TVPs were mixed into the paste mixture by hand which was followed by addition of the colourants. A total of 97.1 g of the formulation was weighed and mixed with the salmon cells. As mentioned earlier, Chinook Salmon (CHSE-214 Cells) in PBS were added into the formulation under the laminar flow hood and the formulation was then wrapped tightly in plastic, vacuum-sealed, and sent for further analyses.

In some embodiments, the formulation was treated and fabricated prior to storing it for further analysis. In fabrication, a total of 97.1 g of the scaffold/formulation was reserved. A falcon tube containing 5.59 g of CHSE-214 cells was held in PBS. The BSC (laminar flow hood) was prepared, and the material along with the cells was brought into the prepared space in the BSC. To open the material and increase the contact surface, a sterile petri dish was utilized. The falcon tube containing the cells was tapped against the material until all the content was transferred. Following this step, the scaffold was gently mixed using hands and the sample was thereby fabricated. Finally, the sample was vacuum sealed and stored until further analyses.

### Trial 2

In trial 2, The plant-based salmon fillet was formulated using the following ingredients:
84 g water
35.5 g mung bean isolate
20 g floss mung bean TVP
36.8 g wheat TVP
28 g sunflower oil
2 g konjac gum
2.5 g tapioca starch
2 g Kappa-carrageenan
6 g 0.5M Ca(OH)₂
1 g salt
0.15 g CaSO₄
90 g Mer AA
1.2 g salmon flavour
0.9 g flavour masker
0.04 g ExBerry brilliant orange (liquid)
0.04 g Astaxanthin
0.15 g Watermelon pink (liquid)
0.6 g Glucone delta lactone

The formulation was treated with Chinook Salmon cells and PBS and the following concentrations were used:
Formulation—61.6 g (97%)
Chinook Salmon (CHSE-214 Cells)—1.9 g (3%)
Total—63.5 g (100%)

The formulation was analysed for Protein, Lipid, Sodium, and Cholesterol content.

### Protocol

All the ingredients were weighed. Out of all the ingredients, mung bean and wheat TVP were soaked in hot water for 30 minutes in separate bowls. Water was then squeezed out from the soaked TVP. All ingredients were mixed, except Ca(OH)₂ and the 2 TVPs, in a thermomixer to form a paste. The paste was then heated in the microwave for 20 seconds, followed by which, 6 g 0.5M Ca(OH)₂ were then added and mixed into the paste in a thermomixer. The shredded TVPs were mixed into the paste mixture by hand, followed by which the colourants were added. A total of 61.6 g of the formulation was weighed and mixed with the salmon cells. As noted earlier, Chinook Salmon (CHSE-214 Cells) in PBS were added into the formulation under the laminar flow hood. The formulation was then shaped into a salmon fillet by hand with gloves and was wrapped tightly in plastic. The fillet was vacuum sealed and frozen overnight. The filet was then sliced into slabs using a cutter, more specifically, the fillet was cut on a 45° angle to the length of the filet; straight down the filet. The paste slices and vegan mayo were stacked with each other. Vegan mayo was added with an offset spatula to get a thin layer. Finally, the salmon fillet was wrapped tightly in plastic, vacuum-sealed, and sent to further analyses.

In some embodiments, the formulation was treated and fabricated prior to storing it for further analysis. A total of 61.6 g of the scaffold/formulation was reserved. A falcon tube containing 1.9 g of CHSE-214 Cells in PBS. The BSC (laminar flow hood) was prepared, and the material along with the cells was brought into the prepared space in the BSC. A sterile petri dish was utilized to open the material and increase the contact surface. The falcon tube containing the cells was tapped against the formulation until all the content was transferred. The scaffold was gently mixed using hands and the sample was fabricated. The formulation was moulded, vacuum-sealed, frozen, cut, and the white lines were added (all the processes were performed in BSC and the instruments were sterilized with ethanol 70%. After the application of the white line, the salmon fillet prototype was wrapped in plastic, vacuum-sealed and stored until analysis.

### Results

The nutritional profile of Trial 1 and Trial 2 of salmon fillet prototype infiltrated with Chinook cells, and results of the analysis are provided in Table 8 below.

TABLE 8

Treatments

| | | | USDA (Chinook |
|---|---|---|---|
| Parameters | Trial 1 | Trial 2 | salmon) |
| Sodium (mg/100 g) | 378 | 352 | 47 |
| Nitrogen (%) | 2.91 | 3.24 | — |
| Protein (%) | 18.18 | 20.28 | 19.9 |
| Fat (%) | 12.7 | 9.43 | 10.4 |
| Cholesterol (mg/100 g) | 6.8 | 7 | 50 |
| Humidity (%) | 63.9 | — | 71.6 |
| Ash (%) | 1.15 | — | 1.33 |

As is clearly evident from the table, the formulation with cells demonstrated protein, lipid, humidity, and ash mostly within 10% deviation compared to the conventional salmon. The contents of sodium and cholesterol are still discrepant in comparison with the conventional counterpart. The cholesterol concentration is relatively higher, and therefore, it cannot be pursued as a parameter. However, the cholesterol content, reaffirmed the role of the Chinook cells as being responsible for adding cholesterol in the formulation.

### Example 23—Plant Based Salmon Prototype—Experiment 2: Cooking and Sensory Analysis of Cooked Salmon Prototype Infiltrated with Fish Cells (CHSE-214) Using Mixing and Vacuum Techniques

### Objective

The objective was to add cells from Chinook salmon to the plant-based salmon prototype, and create the cell-based salmon prototype, and perform the first tasting with the cell-based final product.

The formulation consisted of the following ingredients:
96.5 g Water
20 g Mung Bean TV
35.5 g Isolated mung bean protein (80%)
26.5 g Sunflower oil
2 g Konjac gum
6 g 0.5M Ca(OH)₂
1 g Salt
0.6 g Glucone Delta Lactone
78.5 g MerAA
3 g Tapioca Starch
16.5 g Wheat TVP
0.04 g Exberry Brilliant Orange
0.04 g Astaxanthin
0.15 g Watermelon Pink
0.9 g Flavourcan Masker
1.2 g Salmon Flavour
0.64 g Salmon cells
0.69 g PBS

### Protocol

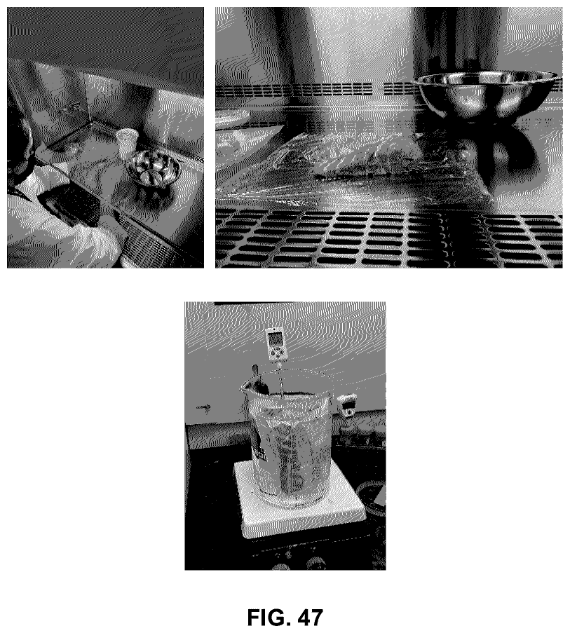

Out of all the ingredients, mung bean and wheat TVP were soaked in hot water for 30 minutes in separate bowls. Water was then squeezed out from the soaked TVP. All ingredients were mixed, except Ca(OH)₂ and the 2 TVPs, in a thermomixer to form a paste. The paste was then heated in the microwave for 20 seconds, and was followed by addition of 6 g 0.5M Ca(OH)₂ and then mixed into the paste in a thermomixer. The shredded TVPs were mixed into the paste mixture by hand. The colourants were then added. Salmon cells in PBS (Chinook salmon cell line—CHSE-214) were added and mixed into the formulation under the laminar flow hood. Specifically, 0.6426 g cells (salmon)+0.6874 g PBS, were added to the formulation. The formulation was then shaped into a salmon filet shape by hand, vacuum-sealed and frozen for 24 h. The filet was brought to the laminar flow hood and then sliced into slabs using a cutter on a 45° angle to the length of the filet; straight down the filet. The salmon prototype slices and vegan mayo were stacked with each other. Vegan mayo was put with an offset spatula to get a thin layer. The salmon prototype was vacuum sealed for a second time and placed into a beaker at a controlled temperature (60° C.) for 1 h. As a final step, before tasting, the sample was cooked for 15 min at 300° F. in a conventional oven. The cell-based salmon prototype preparation, especially cell addition, assembling, and cooking, simulating the Sous Vide is shown in FIG. 47.

### Observations

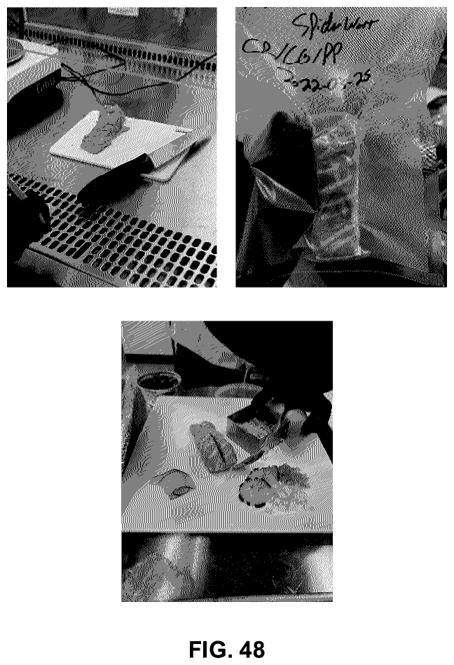

Feel: The chinook salmon cells were incorporated well into the formulations, albeit rendering the samples stickier. There seemed to be little to no difference in terms of the cell-based formulation fabrication, with cutting and white lines, in comparison to the normally controlled formulation other than the fact the sample containing cells was partially stickier than the controlled formulation. FIG. 48 sows the cell-based salmon prototype preparation (especially cell addition, assembling, cooking simulating the Sous Vide, and tasting).

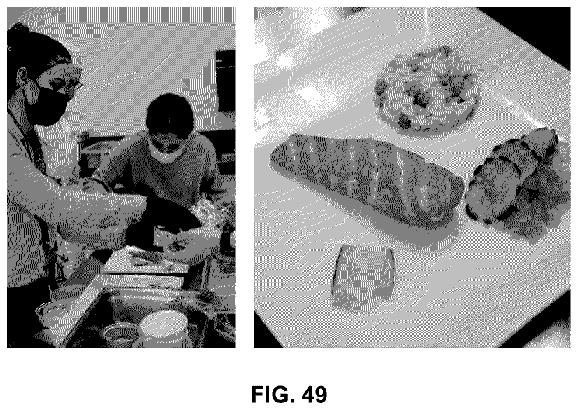

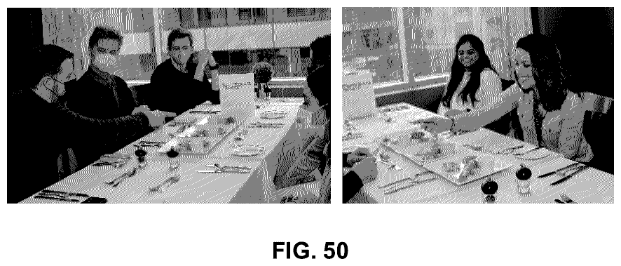

Tasting: The formulation with cells was visually similar to the formulation without cells however, when it was tasted, it appeared moister than the control. The tasting resulted in a split opinion between all participants, with some finding the cell formulation moister and closer to that of salmon, while others preferred the control formulation for the exact opposite reasons. The preparation of the cell-based salmon prototype to be tasted and tasting of the cell-based salmon prototype are shown in FIGS. 49 and 50.

Appearance: There was no change in the appearance of the formulation upon adding the cells, other than the physical stickiness of the formulations increasing. This poses some issues in terms of moulding and mixing of the formulations, however by accommodating the assumed loss, we are able to retain the desired mass of the final product.

### Example 24—Plant Based Salmon Prototype—Experiment 3: Infiltration of C2C12 on the Salmon Prototype and Histology

### Objective

The objective was to infiltrate C2C12 cells, instead of Chinook cells, on the cooked salmon prototype using mixing and vacuum techniques.

### Formulation and Protocol

### A. Cell (C2C12) Production:

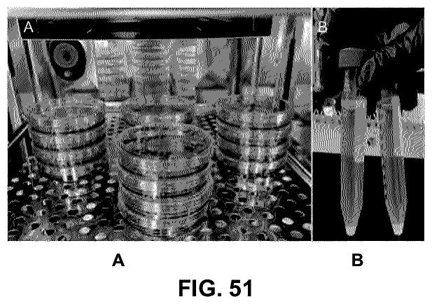

C2C12 was grown as undifferentiated myoblasts in growth medium, GM [DMEM-high glucose no sodium pyruvate (Gibco), 10% FBS (HyClone or Gibco), 1% penicillin/streptomycin, at 37° C. 5% CO₂]. GM was replaced every 2 days, when cells reached close to 80% confluency. First the tube with C2C12 cells was taken from the freezer −80° C. and was thawed at room temperature. The cells were transferred in a Falcon tube and centrifuge for 3 minutes at 1000 rpm. After the supernatant was removed, they were resuspended in the medium and transferred to a sterile petri dish. More media was added and the cells were left in the incubator. After two days, the medium (DMEM) was removed and fresh media was added. When cells were 50-60% confluent they were split 1:4. For the passage of cells, the culture medium was removed and discarded. The cells were rinsed with PBS and 2 mL of 0.05% trypsin was added. The cells were then incubated at 37° C. and 5% CO₂ for 6 minutes. After cells detached, 5 mL of medium was added to the petri dish and the material was transferred to a Falcon tube, and more media was added to the aliquot of cell suspension and finally the cells were collected by gently pipetting and transferred to more cell petri dishes until there were 0.5 g of cells. FIG. 51A shows C2C12 cells line proliferating in DMEM and the suspension of cells after 18 days of proliferation is shown in FIG. 51B.

### B. Formulation and Infiltration of C2C12 Cells in the Salmon Prototype

The formulation was prepared with the following ingredients:
84 g Water
20 g Mung Bean TVP
15 g Isolated mung bean protein (80%)
26 g Sunflower oil
10 g Konjac gum
6 g 0.5M Ca(OH)₂
1 g Salt
0.6 g Glucone Delta Lactone
90 g MerAA
2.5 g Kappa-carrageenan
2 g Tapioca Starch
40 g Wheat TVP
0.04 g Exberry Brilliant Orange
0.04 g Astaxanthin
0.15 g Watermelon Pink
0.9 g Flavourcan Masker
3 g Salmon Flavour

### Protocol

Out of all the ingredients, mung bean and wheat TVP were soaked in hot water for 30 minutes in separate bowls. Water was then squeezed out from the soaked TVP. All ingredients were mixed, except Ca(OH)₂ and the 2 TVPs, in a thermomixer to form a paste. The paste was then heated in the microwave for 20 seconds. A total of 6 g 0.5M Ca(OH)₂ were then added and mixed into the paste in a thermomixer. The shredded TVPs were mixed into the paste mixture by hand. The colourants were then added. Two 5.0 g samples of the formulation were weighed and mixed with the C2C12 cells. Another 5.0 g of the formulation was weighed to be utilized as a control. The formulation was then wrapped tightly in plastic, vacuum-sealed, and sent to further analyses.

The treatment containing cells was fabricated as follows:
The BSC (laminar flow hood) was prepared, and the material along with the cells was brought into the prepared space in the BSC.
A total of 10.0 g (two samples of 5.0 g) of the scaffold/formulation were reserved.
The percentage of cells transferred to the WBF-material (5.0 g) was 10% of the total mass. The mass of cells was determined by the mass difference between an empty falcon tube and a falcon tube with cells after centrifugation after 18 days of cell culture.
A noted earlier, two samples were prepared. Each sample of WBF-material was inoculated with 53.341×106 cells present in 0.5 g of cell suspension.

Additionally, one control sample without cell suspension was prepared too.
All steps were performed in a biosafety cabinet using proper aseptic techniques.
The scaffold was then gently mixed using hands and the sample was fabricated, vacuum-sealed for 24 h and stored until further analysis.
Both samples (the block containing cells and without cells were submerged in formalin) and were maintained for 24-48 h.
Sections were cut from the block to analyze the histology.
For the histology, two sections were cut from each treatment, one from the surface and the other from the center. The sections were allocated into falcon tubes and stored in 70% ethanol.
For ease of reference, the samples for histology were labelled as follows:
WSQWR—Control outer
AXCDZ—Control middle
AKYUL—With cells outer
EZHGB—With cells middle

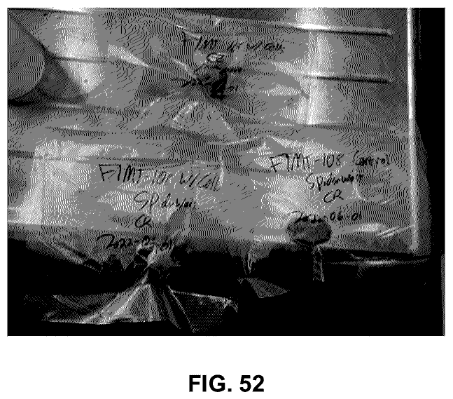

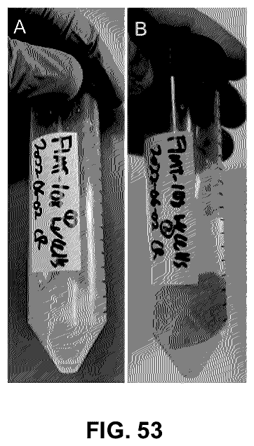

The WBF-material infiltrated with Chinook salmon cells after vacuum process is shown in FIG. 52 and C2C12 cells line inoculated in the WBF-material in formalin is shown in FIG. 53.

### Histology Results and Highlights

In histology analysis, cuts from the surface and middle from control plant-based salmon prototype and cell-based counterparts were analyzed using histology. The dyes used for analysis were Hematoxylin and Eosin, as hematoxylin which has a deep blue-purple colour is capable of staining nucleic acids whereas eosin which is pink in colour is capable of staining proteins nonspecifically. When these dyes are used, in a typical tissue, nuclei are stained blue, and the cytoplasm and also extracellular matrix have varying degrees of pink staining.

The cell-based salmon demonstrated a similar trend when compared to the conventional meat, exhibiting nuclei from C2C12 stained in blue and for the plant-based scaffold varied degrees of pink staining. An evident nuclei staining in 10× magnification was observed for the cell-based salmon prototype in the surface and in the middle of the sample, highlighting the difference between the plant-based and cell-based prototype. Thus the histology revealed the difference between the plant-based and cell-based prototypes.

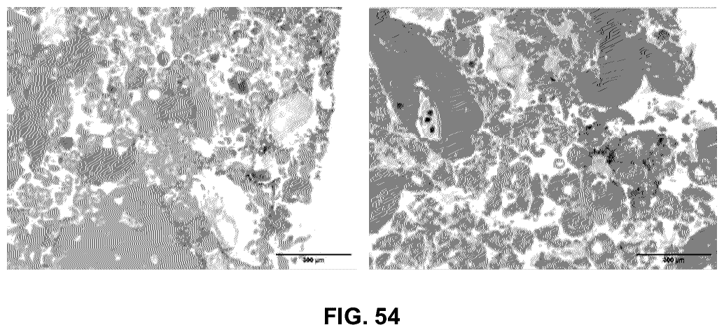

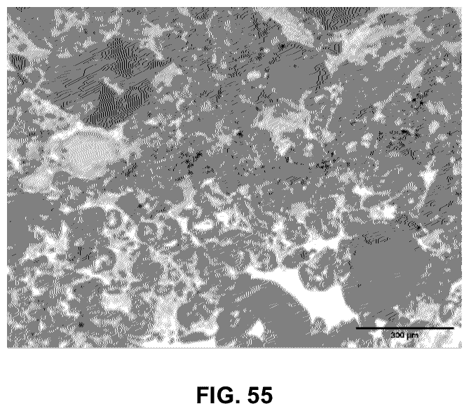

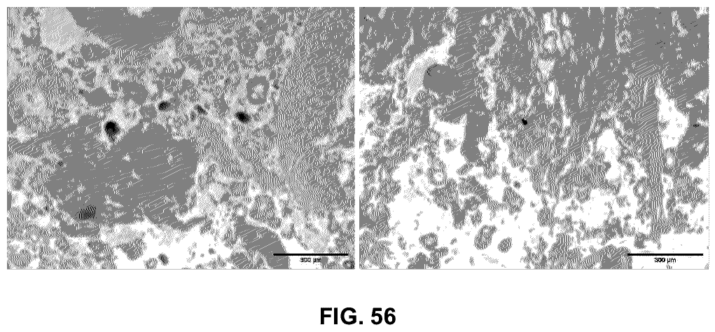

FIG. 54 shows the cell-based salmon prototype infiltrated with C2C12 cells (surface of the sample) at 10× magnification, HE. FIG. 55 shows the Cell-based salmon prototype infiltrated with C2C12 cells (middle of the sample) 10×, HE. FIG. 56 shows the surface and middle of the plant-based salmon prototype (Left and Right respectively) 10×, HE.

### Example 25—Plant Based Salmon Prototype—Experiment 4: Microscopy Analysis of the Salmon Prototype Infiltrated with Chinook Salmon Cells (CHSE-214) Using Combined Techniques—Vacuum and Mixing

The formulation was prepared using the following ingredients:
84 g water
35.5 g mung bean isolate
20 g floss mung bean TVP
26.8 g wheat TVP
30 g sunflower oil
2 g konjac gum
2.5 g tapioca starch
2 g Kappa-carrageenan
6 g 0.5M Ca(OH)₂
1 g salt
0.15 g CaSO₄
102 g Mer AA
1.2 g salmon flavour
0.9 g flavour masker
0.04 g ExBerry brilliant orange (liquid)
0.04 g Astaxanthin
0.15 g Watermelon pink (liquid)
0.6 g Glucone delta lactone

The formulation was treated with Chinook Salmon Cells and PBS and the following concentrations were used: Formulation—47.5 g (79.06%), White Lines—4 g (6.66%), Chinook Salmon (CHSE-214 Cells)—5.43 g (9.04%), PBS—3.15 g (5.24%), with a Total concentration of 60.08 (100%).

### Protocol

All the ingredients were weighed. Out of the ingredients, mung bean and wheat TVP were soaked in hot water for 30 minutes in separate bowls. Water was then squeezed out from the soaked TVP. All ingredients were mixed, except Ca(OH)₂ and the 2 TVPs, in a thermomixer to form a paste. The paste was then heated in the microwave for 20 seconds, and 6 g 0.5M Ca(OH)₂ were then added and mixed into the paste in a thermomixer. The shredded TVPs were mixed into the paste mixture by hand, followed by which the colourants were added. The BSC (laminar flow hood) was prepared, and the material along with the cells was brought into the prepared space in the BSC. A total of 47.5 g of the scaffold/formulation and a falcon tube containing 5.43 g of CHSE-214 Cells in PBS were reserved. A sterile petri dish was utilized to open the material and increase the contact surface. The falcon tube containing the cells was tapped against the formulation until all the content was transferred. The scaffold was gently mixed using hands and the sample was fabricated, and 5 g of the salmon fillets prototype containing the CHSE-214 Cells was reserved. and vacuum sealed for 12 h. The 5 g samples were dissected in 4 (1×1×1 cm3), allocated in falcon tubes containing PFA (paraformaldehyde solution) 4% and maintained for 6 h in the fixing step. The samples for the fluorescence analysis were stained with Hoescht (1:1000, Hoechst: PBS) for microscopy for 30 min, washed again 3× with PBS, and analyzed utilizing a 10× magnification. The samples stained with Hoechst were analyzed in the microscope and examined under a DAPI filter. The images were analyzed in Image J using Cyan dye that stains nuclei, and magenta dye that stains the scaffold. The composite of both images was fabricated and further analyzed.

### Results and Highlights

The addition of cells was facilitated when cells were suspended in PBS; however, this may increase the sodium content of the salmon. The texture of the sample became much more moist upon the addition of cells/PBS. The image seen by microscopy demonstrated visible cells embedded in the sample, located around the scaffolding of the sample.

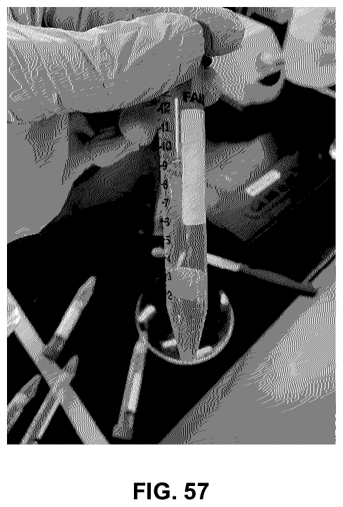

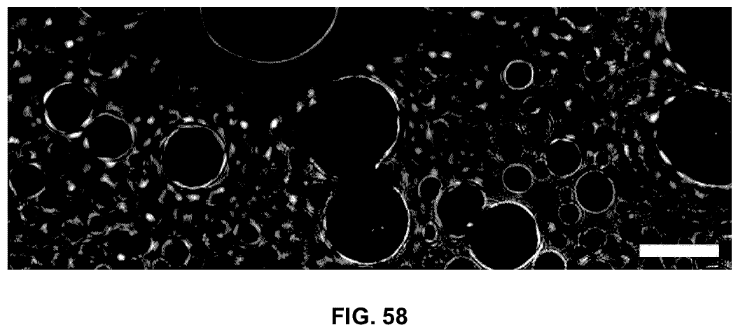

FIG. 57 shows the starting material with the cells suspended in PFA. FIG. 58 shows the seeded scaffold at a scale: 100 μm, wherein cyan represents nuclei, magenta represents scaffold. Seeded scaffold is also stained with Hoescht (1:1000) under DAPI filters observed under 10× magnification.

From the above examples and experimental data, a person skilled in the art would appreciate that a variety of seeded bio-scaffolds using different cell types and ingredients can be created using the techniques described in the present invention. Apart from the specific examples and products described in the application which substantiates and validates the infiltration technique, a person skilled in the art would be able to comprehend that other meat products, lab-grown meat products and/or plant-based recipes could very well be produced using the technique and methods discussed in the application.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the elements that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A method of preparing a seeded biomaterial scaffold, the method comprising:
combining a biomaterial scaffold and a plurality of cells to provide a mixture; and
applying a pressure to the mixture to thereby cause the plurality of cells to homogeneously distribute throughout the scaffold,
thereby forming the seeded biomaterial scaffold.

2. The method of claim 1, wherein a positive pressure is applied by mixing the biomaterial scaffold and the plurality of cells using one or more syringes, or by using mixing methods such as stirring, beating, blending, cutting in, whisking, folding or emulsifying.

3. The method of claim 1, wherein a decrease in pressure is applied wherein the pressure is decreased between 0 to 101.3 kPa below atmospheric pressure to homogeneously distribute the plurality of cells throughout the scaffold.

4. The method of claim 3, wherein the decrease in pressure further distributes flavorings and/or colourants throughout the scaffold.

5. The method of claim 2, wherein the positive pressure is applied at 0.001 to 900 MPa.

6. The method of claim 1, wherein the pressure is decreased at 0 to 100 kPa relative to atmospheric pressure.

7. The method of any one of claims 1 to 6, wherein the plurality of cells comprise a homogeneous or a heterogeneous population of cells.

8. The method of claim 7, wherein the homogeneous or heterogeneous population of cells comprise muscle cells, fat cells, connective tissue cells, cartilage, bone, epithelial, or endothelial cells, or any combinations thereof.

9. The method of claim 3, 4 or 6, wherein the mixture is vacuum sealed for 30 minutes to about 7 days.

10. The method of claim 3, 4, 6 or 9, wherein the mixture is maintained under vacuum at a temperature of about 0° C. to about 100° C. and/or thermically treated at a temperature of about 30° C. to 150° C.

11. The method of claim 10, wherein the mixture is maintained under vacuum at a temperature of about 0° C. to about 10° C.

12. The method of any one of claims 1 to 11, wherein the biomaterial scaffold comprises one or more crosslinkable components.

13. The method of claim 12, wherein the one or more crosslinkable components comprise cellulose, a cellulose derivative such as methylcellulose, carboxymethylcellulose, hydroxypropylcellulose, hydroxypropyl methyl cellulose, hydroxyethyl methyl cellulose, ethyl cellulose and dissolved or regenerated cellulose, leguminous proteins (such as soy protein, mung bean protein, pea protein, kidney bean protein, lupin protein, and chickpea protein), cereal proteins (such as wheat protein, rice protein, and corn protein), oil seed proteins (such as peanut protein, sunflower protein, canola protein, flax seed protein, sesame protein), fungi protein (such as mycoprotein, yeast, mushrooms), and canola oil protein, different classes of food hydrocolloids comprising one or more crosslinkable components such as plant-derived hydrocolloids (including but not limited to plant exudates; such as acacia gum, gum arabic, tragacanth, khaya gum, karaya gum, ghatti gum, pectin, inulin, chicle gum, konjac glucomannan; seed gums such as guar gum, locust bean gum, fenugreek gum, cassia seed gum, basil seed gum, mesquite seed gum, oat gum, lesquerella, fendleri gum, rye gum, Psyllium, premcem gum, starch, amylase, cellulose, tamarind seed gum, seaweed—agar-agar, carrageenan, alginic acid, sodium alginate, furcellaran, ulvan, flucoidan, laminarin, red alga, xylan) animal-derived hydrocolloids (including but not limited to gelatin, chitin, and chitosan), hydrocolloids from microbial sources—fermentation (microbial exudates—xanthan, dextran, curdlan, scleroglucan, gellan gum, pullulan, tara guma, spruce gum, and baker's yeast glycan), and chemically modified plant-derived hydrocolloids—synthetic gums (including but not limited to modified starch—hetastarch, starch acetates, startch phosphates) hyaluronic acid, elastin, fibrin, fibrinogen, or the like, or any combination thereof. The aforementioned components (including proteins) can be cross-linked using chemical, physical, or enzymatic techniques, for example, using glutaraldehyde, glyoxal, genipin, diimidoesters-dimethyl suberimidate, 3,3′-dithiobispropionimidate, sorbitol, glycerol, hexamethylene diisocyanate (HMDC), calcium chloride, calcium hydroxide, monovalent ions such as H+, Na+, K+, Cs+, Rb+, and I−, multivalent ions such as Mg2+, Ca2+, Ba2+, Fe2+, Cu2+, Zn2+, Fe3+ and A13+, divalent ion salts, acids (such as citric acid, tannic acid, malic acid, and glutamic acid), enzymes (such as tranglutaminase, oxidoreductases), phenolic acids, flavonoids, glucono delta lactone, high pressure, irradiation, optical radiation, ionizing radiation, and the like.

14. The method of claim 12 or 13, wherein the biomaterial scaffold is crosslinked.

15. The method of claim 12 or 13, further comprising crosslinking the mixture with a crosslinker selected from transglutaminase, glutaraldehyde, riboflavin, formalin, formaldehyde, transglutaminase, EDC (1-ethyl-3-(3-dimethylaminopropyl)carbodiimide hydrochloride), calcium chloride, magnesium chloride, citric acid, glycine, divinylsulfone, PVA, EVA, glyoxal, carrageenan.

16. The method of claim 15, wherein the crosslinking of the mixture is performed prior to the applying of the pressure.

17. The method of claim 15, wherein the crosslinking of the mixture is performed after the applying of the pressure.

18. The method of claim 15, wherein the crosslinking of the mixture is performed simultaneously with the applying of the pressure.

19. The method of any one of claims 1 to 18, wherein the plurality of cells are provided in a cell culture media.

20. The method of claim 19, wherein the cell culture media is Dulbecco's Modified Eagle Medium (DMEM).

21. The method of claim 19 or 20, wherein the cell culture media comprises at least one growth factor.

22. The method of claim 21, wherein the growth factor comprises fetal bovine serum (FBS).

23. The method of any one of claims 1 to 22, wherein the cell culture media comprises an antibiotic.

24. The method of claim 23, wherein the antibiotic comprises penicillin-streptomycin.

25. The method of any one of claims 1 to 24, wherein the cell culture media comprises an antifungal.

26. The method of claim 25, wherein the antifungal comprises Amphotericin B.

27. The method of any one of claims 1 to 26, wherein the scaffold comprises a decellularized plant or fungal tissue.

28. The method of claim 27, wherein the decellularized plant or fungal tissue is a mercerized plant or fungal tissue.

29. The method of claim 27 or 28, wherein the plant or fungal tissue comprises an apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesil) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, a rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, a pear (Pomaceous) tissue, a heart of the palm (Bactris gasipaes) tissue, an artichoke (Cynara cardunculus var. scolymus) tissue, a lotus roots (Nelumbo nucifera), banana blossom (Musa acuminata), Bamboo shoot (Bambusa vulgaris and Phyllostachys edulis), or any combination thereof.

30. The method of any one of claims 1 to 29, further comprising preparing the biomaterial scaffold by mercerizing a plant or fungal tissue.

31. The method of claim 30, wherein the mercerizing of the plant or fungal tissue comprises mixing the plant or fungal tissue with a bicarbonate solution.

32. The method of claim 31, wherein the bicarbonate solution is a 10% sodium bicarbonate solution.

33. The method of any one of claims 30 to 32, wherein the mercerizing of the plant or fungal tissue comprises bleaching the plant or fungal tissue with a peroxide solution.

34. The method of claim 33, wherein the peroxide solution is an about 9% or about 15% hydrogen peroxide stock solution.

35. The method of any one of claims 30 to 34, wherein the preparing of the biomaterial scaffold further comprises decellularizing the plant of fungal tissue prior to the mercerization.

36. The method of claim 29, wherein the decellularizing of the plant or fungal tissue comprises mixing the plant or fungal tissue with a sodium dodecyl sulfate (SDS) solution.

37. The method of claim 36, wherein residual SDS is removed using a divalent salt solution.

38. The method of claim 37, wherein the divalent salt solution is an MgCl₂ solution or a CaCl₂) solution.

39. The method of claim 38, wherein the divalent salt solution comprises the divalent salt at a concentration of about 50 mM to about 150 mM.

40. The method of any one of claims 1 to 39, further comprising directionally freezing the biomaterial scaffold.

41. The method of any one of claims 1 to 40, further comprising dividing the biomaterial scaffold or the seeded biomaterial scaffold into a plurality of strips, shapes and/or thicknesses of the biomaterial scaffold, and adhering the strips, shapes and/or thicknesses of the biomaterial scaffold together to form a layered biomaterial scaffold or a layered seeded biomaterial scaffold.

42. The method of claim 41, wherein the plurality of strips shapes and/or thicknesses of the biomaterial scaffold are adhered together using a crosslinker.

43. The method of claim 42, wherein the crosslinker comprises a transglutaminase.

44. The method of any one of claims 1 to 43, wherein one or more colouring agents is added to the biomaterial scaffold.

45. The method of any one of claims 1 to 44, wherein the biomaterial scaffold is pre-treated with a flavoring and/or to provide a desired flavour.

46. The method of any one of claims 1 to 45, which is kitchen-safe.

47. A seeded biomaterial scaffold produced by the method of any one of claims 1 to 46.

48. Use of the seeded biomaterial scaffold of claim 47 for the production of a cultured meat product.

49. Use of the seeded biomaterial scaffold of claim 47 for the production of a vegan meat product.
